(19) United States
(12) Patent Application Publication
(10) Pub. No.: US 2016/0176819 A1
(43) Pub. Date: Jun. 23, 2016

(54) ASYMMETRICALLY CHARGED NIR FLUOROPHORES

(71) Applicant: Washington University, St. Louis, MO (US)

(72) Inventors: Mikhail Berezin, St. Louis, MO (US); Natalia Zhegalova, St. Louis, MO (US); Haiying Zhou, St. Louis, MO (US)

(21) Appl. No.: 14/973,104

(22) Filed: Dec. 17, 2015

Related U.S. Application Data
(60) Provisional application No. 62/094,181, filed on Dec. 19, 2014.

(51) Int. Cl. C07D 209/12; C07K 14/765; C12N 9/36; A61K 49/00; A61K 47/48; C09B 23/16; C07K 16/18

(52) U.S. Cl. CPC C07D 209/12; C09B 23/164; C07K 14/765; C07K 16/18; A61K 49/0052; A61K 47/48061; C12N 9/2462; C12Y 302/01017

(57) ABSTRACT

The invention encompasses asymmetrically charged fluorescent cyanine dyes and methods of using such dyes. In particular, the invention encompasses near infrared polymethine cyanine dyes.

### CROSS REFERENCE TO RELATED APPLICATIONS

This application claims the benefit of U.S. Provisional Application No. 62/094,181, filed Dec. 19, 2014, the disclosure of which is hereby incorporated by reference in its entirety.

### GOVERNMENTAL RIGHTS

The invention was made with government support under Grant No. HHSN268201000046C awarded by the National Heart Lung and Blood Institute. The government has certain rights in this invention.

### FIELD OF THE INVENTION

The invention encompasses stable near-infrared cyanine dyes that are asymmetrically charged and methods of using such dyes.

### BACKGROUND OF THE INVENTION

Optical imaging is an important method in diagnostics to overcome the problems associated with radiation and MRI. Despite the benefits, optical imaging is challenging due to the lack appropriate fluorescent dyes. The excellent safety profile of the NIR heptamethine cyanine fluorochrome indocyanine green (ICG) in humans has spurred interest in the development of ICG derivatives, including Cy dyes for in vivo molecular imaging by NIR optical methods. Cyanine dyes that absorb and emit light in the near-infrared (NIR) wavelengths have been widely used for labeling biomolecules including antibodies, DNA probes, avidin, streptavidin, lipids, biochemical analogs, peptides, nanoparticles and drugs, as well as for a variety of applications including DNA sequencing, DNA microarray, Western blotting, flow cytometry analysis, and protein microarrays to name a few. However, most of the commercially available dyes showed low brightness due to self-aggregation and quenching after conjugation. This requires high dosage of expensive probes (antibodies, nanoparticles, proteins, polypeptides) to be used to be seen. Thus, there is a need for novel dyes that demonstrate improved properties.

### SUMMARY OF THE INVENTION

In an aspect, the disclosure provides an asymmetrically charged fluorescent cyanine dye comprising Formula (I):

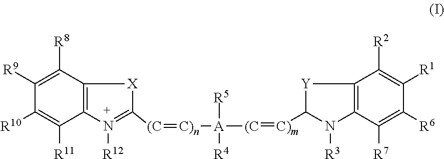

wherein:
A is selected from the group consisting of a carbon, a carbocyclic ring, and a heterocyclic ring;
X and Y are independently selected from the group consisting of a heteroatom, an alkyl group, an alkenyl group, and an alkynyl group;
n and m are independent integers from 1 to about 5;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R², R⁴, R⁵, R⁶, R⁷, R⁸, R¹⁰ and R¹¹ are independently selected from the group consisting of functional group, hydrogen, hydrocarbyl, and substituted hydrocarbyl; and
R³ and R¹² are independently selected from the group consisting of hydrocarbyl, and substituted hydrocarbyl.

In another aspect, the disclosure provides an asymmetrically charged fluorescent cyanine dye comprising Formula (II):

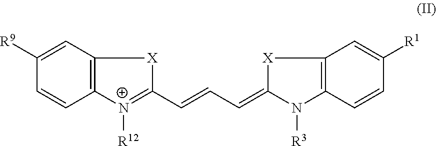

wherein:
X is selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group; and
R³ and R¹² are independently selected from the group consisting of hydrocarbyl, and substituted hydrocarbyl.

In still another aspect, the disclosure provides an asymmetrically charged fluorescent cyanine dye comprising Formula (III):

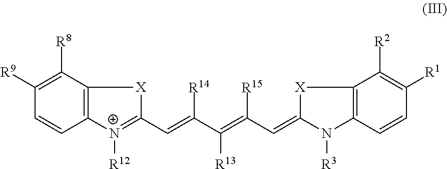

wherein:
X is selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R² and R⁸ are independently selected from the group consisting of functional group, hydrogen, hydrocarbyl, and substituted hydrocarbyl; and
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

In still yet another aspect, the disclosure provides an asymmetrically charged fluorescent cyanine dye comprising Formula (IV):

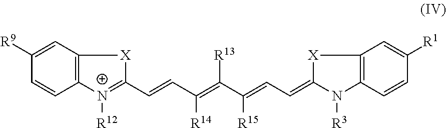

wherein:
X is selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

In a different aspect, the disclosure provides an asymmetrically charged fluorescent cyanine dye comprising Formula (I), (II), (III) or (IV) conjugated to a biomolecule.

In another different aspect, the disclosure provides a method of use of an asymmetrically charged fluorescent cyanine dye comprising Formula (I), (II), (III) or (IV) comprising administering the asymmetrically charged fluorescent cyanine dye to a subject and detecting the asymmetrically charged fluorescent cyanine dye in the subject.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the analysis of fluorescent nanoparticles after conjugation. Negatively charged free dye quickly migrates to anode and leaves the gel. Conditions: 2% agarose gel, 160 V, (30 min) Well 1: LS755-NHS 0.5% to amine. Well 2: LS755 0. 5% to amine. Well 3, NPs non labeled.

FIG. 2 depicts a graph showing that fluorescent nanoparticles do not show aggregation (absence of the H-band) and demonstrate high fluorescence quantum yield (10-34%) depending on the loading.

FIG. 3 depicts images of RAW 264.7 cells with LS755-NP conjugates.

FIG. 4 depicts a graph of a viability study with alveolar macrophages that shows low cell toxicity of LS755-NP conjugates.

FIG. 5A, FIG. 5B and FIG. 5C depict NIR images of mice (FIG. 5A) immediately after and (FIG. 5B) 72 h and (FIG. 5C) 168 hours post-injection. Bright fluorescence remains visible in lungs. Biodistribution study shows accumulation of the fluorescence primarily in lungs. Pearl imager (Li-Cor).

FIG. 6A, FIG. 6B and FIG. 6C depict images of the biodistribution of LS755-NP conjugates. (FIG. 6A) Biodistribution showing strongest fluorescence in the lung, (FIG. 6B) lung tissue histology (H&E), and (FIG. 6C) NIR fluorescence imaging of lung tissue overlapped with DIC images. Olympus microscope.

FIG. 7 depicts a graph of the absorption and emission spectra of LS822 in water.

FIG. 8 depicts a graph demonstrating the method of assessing the brightness of fluorescent antibodies using compensation beads. Left peak correspond to a negative beads with no affinity to antibodies. Right peak corresponds to the beads with high affinity to antibodies. At higher loading antibodies saturate binding sites on the beads. The brighter the labeled antibody the larger the separation between the peaks. The data with LS822-IgG show the value of ˜1000 fluorescent units, while a typical value for other labeled antibodies in this spectral channel is several hundred fluorescent units. These data show excellent brightness of the LS-822-IgG as a model.

FIG. 9 depicts a graph showing better brightness of the dye labeled antibodies with asymmetric fluorophores. Flow cytometry results using compensation beads. LS822-IgG shows stronger fluorescence than IR650-IgG.

FIG. 10A and FIG. 10B depict a schematic of the principle of the near-infrared dye for minimum self-aggregation upon conjugation to proteins. Stacking causes dye self-aggregation; charged ends prevent dye from aggregating.

FIG. 11A depicts the structure of LS601 and FIG. 11B depicts the structure of LS755.

FIG. 12 depicts an image of a SDS-PAGE of LS755 conjugates; excitation/emission, 735/800 nm. From left to right: LS755-BSA, LS755-Lz (two lanes of different well loading), LS755-IgG, LS755-PEG, LS755-NHS (two lanes of different well loading).

FIG. 13A depicts the charge distribution of LS601 and FIG. 13B depicts the charge distribution of LS755 pseudoconjugates. The charges are given using a 0.5 Å sphere. Calculated using MM3/AM1 parameters implemented in Cache 5.0 modeling package.

FIG. 14A depicts graphs of the absorption spectra of IgG antibody labeled with LS601 and FIG. 14B depicts graphs of the absorption spectra of IgG antibody labeled with LS755. LS601 shows the presence of an H-band that is almost negligible in LS755-conjugates.

FIG. 15 depicts a graph of the absorption of LS755-lysozyme and LS-BSA conjugates showing no presence of H-bands.

FIG. 16 depicts a graph showing fluorescence decays of LS601-IgG and LS755-IgG conjugates (excitation/emission 740/790 nm).

FIG. 17A, FIG. 17B and FIG. 17C depict a comparison of the fluorescence intensity of LS822-IgG conjugate at different degrees of labeling. (FIG. 17A) Flow cytometry of beads with LS822-IgG (DOL 1.81). (FIG. 17B) Flow cytometry of beads with LS822-IgG (DOL 3.15). (FIG. 17C) Difference of means comparison between two DOLs. Excitation 637 nm, emission 660/20 nm.

### DETAILED DESCRIPTION OF THE INVENTION

Self-aggregation of NIR dyes even at low concentrations poses a considerable challenge in preparing sufficiently bright molecular probes for in vivo imaging. Such self-aggregation leads to severe quenching and low brightness of the targeted probe. To address this problem, the inventors have designed a novel type of dye with an asymmetrical distribution of charge. Asymmetrical distribution prevents the chromophores from stacking, resulting in very high brightness of the probes. Accordingly, the present invention provides asymmetrically charged cyanine dyes and methods of producing such dyes that can be used for imaging, biomedical, and analytical applications.

### I. Asymmetrically Charged Fluorescent Cyanine Dyes

The compounds of the invention comprise asymmetrically charged fluorescent cyanine dyes having two quaternized nitrogen atoms linked by a polymethine chain with or without a cyclic group centrally located within the chain. The nitrogen atoms are each independently part of a heteroaromatic ring. Non-limiting examples of heteroaromatic rings include imidazole, pyridine, pyrrole, quinoline, and thiazole. By “asymmetrically charged” is meant that the sum of the charges on the heteroaromatic ring on one side of the compound is not equal to the sum of the charges on the heteroaromatic ring on the other side of the compound. In one embodiment, the heteroaromatic ring on one side of the compound comprises a highly positive charge and the heteroaromatic ring on the other side of the compound comprises a neutral charge. In another embodiment, the heteroaromatic ring on one side of the compound comprises a highly negative charge and the heteroaromatic ring on the other side of the compound comprises a neutral charge. In still another embodiment, the heteroaromatic ring on one side of the compound comprises a highly negative charge and the heteroaromatic ring on the other side of the compound comprises a highly positive charge. The charge may be located on one or more of R¹, R², R³, R⁶, R⁸, R⁹, R¹⁰ and R¹¹.

In one alternative of this embodiment, the compound comprises an asymmetrically charged cyanine dye having Formula (I):

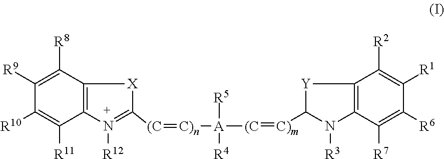

wherein:
A is selected from the group consisting of a carbon, a carbocyclic ring, and a heterocyclic ring;
X and Y are independently selected from the group consisting of a heteroatom, an alkyl group, an alkenyl group, and an alkynyl group;
n and m are independent integers from 1 to about 5;
R¹ and R⁹ are independently selected from the group consisting of a highly charged group and a neutral group with the proviso that at least one of R¹ and R⁹ is a highly charged group;
R², R⁴, R⁵, R⁶, R⁷, R⁸, R¹⁰ and R¹¹ are independently selected from the group consisting of functional group, hydrogen, hydrocarbyl, and substituted hydrocarbyl; and
R³ and R¹² are independently selected from the group consisting of hydrocarbyl, and substituted hydrocarbyl.

In certain embodiments compounds correspond to Formula (I), wherein:
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R², R⁴, R⁵, R⁶, R⁷, R⁸, R¹⁰ and R¹¹ are independently selected from the group consisting of functional group, hydrogen, hydrocarbyl, and substituted hydrocarbyl; and
A, X, Y, n, m, R³ and R¹² are as described above.

In one embodiment compounds correspond to Formula (I), wherein:
R² and R⁸ are independently selected from the group consisting of a highly positive group, a highly negative group, and a neutral group with the proviso that at least one of R² and R⁸ is a highly positive group or a highly negative group and that R² and R⁸ are not both a highly positive group or both a highly negative group;
R¹, R⁴, R⁵, R⁶, R⁷, R⁹, R¹⁰ and R¹¹ are independently selected from the group consisting of functional group, hydrogen, hydrocarbyl, and substituted hydrocarbyl; and
A, X, Y, n, m, R³ and R¹² are as described above.

In another embodiment compounds correspond to Formula (I), wherein:
R⁶ and R¹⁹ are independently selected from the group consisting of a highly positive group, a highly negative group, and a neutral group with the proviso that at least one of R⁶ and R¹⁰ is a highly positive group or a highly negative group and that R⁶ and R¹⁰ are not both a highly positive group or both a highly negative group;
R¹, R², R⁴, R⁵, R⁷, R⁸, R⁹, and R¹¹ are independently selected from the group consisting of functional group, hydrogen, hydrocarbyl, and substituted hydrocarbyl; and
A, X, Y, n, m, R³ and R¹² are as described above.

In still another embodiment compounds correspond to Formula (I), wherein:
R⁷ and R¹¹ are independently selected from the group consisting of a highly positive group, a highly negative group, and a neutral group with the proviso that at least one of R⁷ and R¹¹ is a highly positive group or a highly negative group and that R⁷ and R¹¹ are not both a highly positive group or both a highly negative group;
R¹, R², R⁴, R⁵, R⁶, R⁸, R⁹, and R¹⁰ are independently selected from the group consisting of functional group, hydrogen, hydrocarbyl, and substituted hydrocarbyl; and
A, X, Y, n, m, R³ and R¹² are as described above.

In an exemplary embodiment compounds correspond to Formula (I) wherein:
X and Y are independently selected from the group consisting of sulfur, selenium, oxygen, carbon, nitrogen and C(CH₃)₂;
n and m are independent integers from 1 to 3;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R², R⁴, R⁵, R⁶, R⁷, R⁸, R¹⁰ and R¹¹ are independently selected from the group consisting of functional group, hydrogen, hydrocarbyl, and substituted hydrocarbyl; and
R³ and R¹² are independently selected from the group consisting of hydrocarbyl, and substituted hydrocarbyl.

In one alternative embodiment, the compound encompasses an asymmetrically charged trimethine cyanine dye having Formula (II):

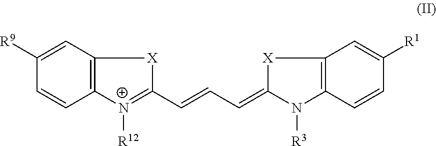

wherein:
X is selected from the group consisting of a heteroatom, an alkyl group, an alkenyl group, and an alkynyl group;
R¹ and R⁹ are independently selected from the group consisting of a highly charged group and a neutral group with the proviso that at least one of R¹ and R⁹ is a highly charged group; and
R³ and R¹² are independently selected from the group consisting of hydrocarbyl, and substituted hydrocarbyl.

In certain embodiments compounds correspond to Formula (II) wherein:
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group; and
X, R³ and R¹² are as described above.

In an embodiment for compounds having Formula (II), R¹ or R⁹ is a highly negative group and R⁹ or R¹ is a neutral group, respectively, and X, R³ and R¹² are as described above.

In another embodiment for compounds having Formula (II), R¹ or R⁹ is a highly positive group and R⁹ or R¹ is a neutral group, respectively, and X, R³ and R¹² are as described above.

In still another embodiment for compounds having Formula (II), R¹ or R⁹ is a highly positive group and R⁹ or R¹ is a highly negative group, respectively, and X, R³ and R¹² are as described above.

In an exemplary embodiment for compounds having Formula (II), X is selected from the group consisting of sulfur, selenium, oxygen, carbon, nitrogen and C(CH₃)₂; and R¹, R³, R⁹, and R¹² are as described above.

In still another alternative of the invention, the compound is an asymmetrically charged pentamethine cyanine dye having Formula (III):

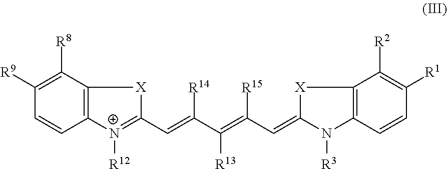

wherein:
X is selected from the group consisting of a heteroatom, an alkyl group, an alkenyl group, and an alkynyl group;
R¹ and R⁹ are independently selected from the group consisting of a highly charged group and a neutral group with the proviso that at least one of R¹ and R⁹ is a highly charged group;
R² and R⁸ are independently selected from the group consisting of functional group, hydrogen, hydrocarbyl, and substituted hydrocarbyl; and
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

In certain embodiments compounds correspond to Formula (III) wherein:
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group; and
X, R², R³, R⁸, R¹², R¹³, R¹⁴ and R¹⁵ are as described above.

In an embodiment for compounds having Formula (III), R¹ or R⁹ is a highly negative group and R⁹ or R¹ is a neutral group, respectively, and X, R², R³, R⁸, R¹², R¹³, R¹⁴ and R¹⁵ are as described above.

In another embodiment for compounds having Formula (III), R¹ or R⁹ is a highly positive group and R⁹ or R¹ is a neutral group, respectively, and X, R², R³, R⁸, R¹², R¹³, R¹⁴ and R¹⁵ are as described above.

In still another embodiment for compounds having Formula (III), R¹ or R⁹ is a highly positive group and R⁹ or R¹ is a highly negative group, respectively, and X, R², R³, R⁸, R¹², R¹³, R¹⁴ and R¹⁵ are as described above.

In another embodiment, for compounds having Formula (III), X is selected from the group consisting of sulfur, selenium, oxygen, carbon, nitrogen and C(CH₃)₂; and R¹, R², R³, R⁸, R⁹, R¹², R¹³, R¹⁴ and R¹⁵ are as described above.

In yet another embodiment, R¹ is a neutral group, R⁹ is a highly negative group; R³ and R¹² are (CH₂)ₙSO₃H, n is an integer from 1 to about 5; and R², R⁸, R¹³, R¹⁴ and R¹⁵ are H.

In yet still another embodiment, R¹ is a highly negative group, R⁹ is a neutral group; R³ and R¹² are (CH₂)ₙSO₃H, n is an integer from 1 to about 5; and R², R⁸, R¹³, R¹⁴ and R¹⁵ are H.

In still another alternative of the invention, the compound is an asymmetrically charged heptamethine cyanine dye having Formula (IV):

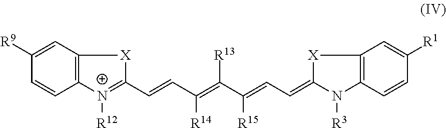

wherein:
X is selected from the group consisting of a heteroatom, an alkyl group, an alkenyl group, and an alkynyl group;
R¹ and R⁹ are independently selected from the group consisting of a highly charged group and a neutral group with the proviso that at least one of R¹ and R⁹ is a highly charged group;
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

In certain embodiments compounds correspond to Formula (IV) wherein:
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group; and
X, R³, R¹², R¹³, R¹⁴ and R¹⁵ are as described above.

In an embodiment for compounds having Formula (IV), R¹ or R⁹ is a highly negative group and R⁹ or R¹ is a neutral group, respectively, and X, R³, R¹², R¹³, R¹⁴ and R¹⁵ are as described above.

In another embodiment for compounds having Formula (IV), R¹ or R⁹ is a highly positive group and R⁹ or R¹ is a neutral group, respectively, and X, R³, R¹², R¹³, R¹⁴ and R¹⁵ are as described above.

In still another embodiment for compounds having Formula (IV), R¹ or R⁹ is a highly positive group and R⁹ or R¹ is a highly negative group, respectively, and X, R³, R¹², R¹³, R¹⁴ and R¹⁵ are as described above.

In another embodiment, for compounds having Formula (IV), X is selected from the group consisting of sulfur, selenium, oxygen, carbon, nitrogen and C(CH₃)₂; and R¹, R³, R⁹, R¹², R¹³, R¹⁴ and R¹⁵ are as described above.

In yet another embodiment, R¹ is a neutral group, R⁹ is a highly negative group; R³ and R¹² are (CH₂)ₙSO₃H, n is an integer from 1 to about 5; and R¹³, R¹⁴ and R¹⁵ are H.

In yet still another embodiment, R¹ is a highly negative group, R⁹ is a neutral group; R³ and R¹² are (CH₂)ₙSO₃H, n is an integer from 1 to about 5; and R¹³, R¹⁴ and R¹⁵ are H.

By “highly charged group” is meant a group that has a strong negative or strong positive charge such that the group is a highly negative group or a highly positive group, respectively. Highly negative group and highly positive group are defined below. In an embodiment, the strong charge may be delocalized and become less strong. Such an embodiment is suitable for the invention.

By “highly negative group” is meant a group that has gained one or more electrons giving it a net negative charge. A highly negative group may also be referred to as an anion. Non-limiting examples of a highly negative group includes a sulfonate group (—SO₃), a phosphate group (—PO₄³⁻), a nitro group (—NO₂) and a carboxyl group (—COO). In a specific embodiment, the highly negative group is a sulfonate group (—SO₃). In another specific embodiment, the highly negative group is a phosphate group (—PO₄³⁻).

By “highly positive group” is meant a group that has lost one or more electrons giving it a net positive charge. A highly positive group may also be referred to as a cation. Non-limiting examples of a highly positive group includes an amine group (—NH₃), a primary amino group, a quaternary ammonium group, an alkylated quaternary amine (i.e. —N(CH₃)₃), or a guanidinium group. In a specific embodiment, the highly positive group is an amine group (—NH₃).

By “neutral group” is meant a group with an equal numbers of protons and electrons, in which case their charges cancel out, yielding a net charge of zero, thus making the group neutral. In a specific embodiment, the neutral group is selected from the group consisting of hydrogen, alkyl, amide, amide, carboxylic acid, azide, alkyne, and hydrazine. The neutral group may be used for conjugation of a biomolecule, described below.

For each of the foregoing embodiments, a benzenindole ring may be used in place of the indole as show in Formula (V), wherein A, X, Y, n, m, R¹, R³, R⁵, R⁹ and R¹² are as described above.

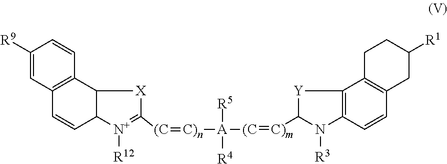

For each of the foregoing embodiments, the cyanine dyes of the invention may include one or more reactive groups for coupling the dye compound to a biomolecule. In one embodiment, one or both R³ and R¹² groups may include a reactive group for coupling the dye compound to a biomolecule. In another embodiment, the R⁵ or R¹³ group may include a reactive group for coupling the dye compound to a biomolecule. In still another embodiment, one or both R³ and R¹² groups and the R⁵ or R¹³ group may include a reactive group for coupling the dye compound to a biomolecule. In still yet another embodiment, the R¹ group or the R⁹ group may include a reactive group for coupling the dye compound to a biomolecule. In different embodiments, one or more of R¹, R², R⁶, R⁷, R⁸, R⁹, R¹⁰, or R¹¹ may include a reactive group for coupling the dye compound to a biomolecule. Suitable non-limiting examples of biomolecules include antigens, steroids, vitamins, drugs, haptens, metabolites, toxins, environmental pollutants, antibodies, DNA, RNA, peptides, proteins, siRNA, miRNA, carbohydrates, lipids, small molecules, and nanoparticles. In an exemplary embodiment, the biomolecule is a peptide. In another exemplary embodiment, the biomolecule is a protein. In still another exemplary embodiment, the biomolecule is a nanoparticle. The biomolecule may be coupled to the dye compound by methods generally known in the art or by methods described herein.

Exemplary non-limiting examples of cyanine dyes of the invention are shown in Table A.

TABLE A

| Compound | |
|---|---|
| Number | Structure |
| LS755 (Cy7 type) | 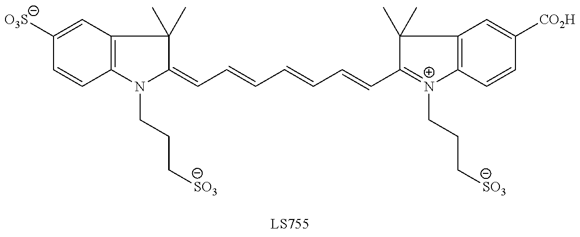 |
| LS822 (Cy5 dye) | 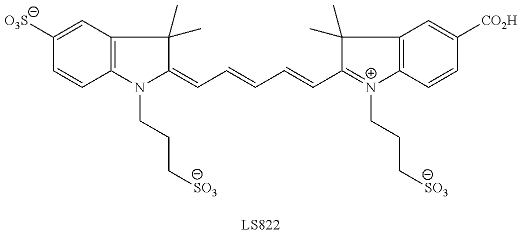 |
| (Cy3 dye) | 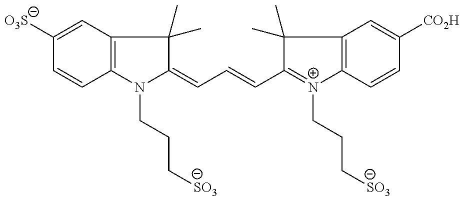 |
| Additional example of Cy 7 with netative charge | 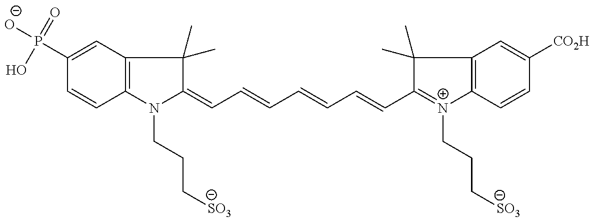 |
| Additional example Cy7 with positive charge | 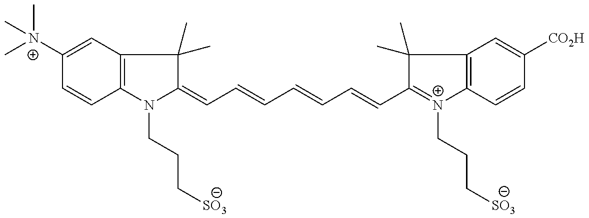 |

The fluorescent cyanine dyes generally have absorption spectra ranging from about 500 nm to about 1000 nm. In an exemplary embodiment, the absorption spectrum is from about 700 nm to about 900 nm. In certain embodiments, the absorption spectra is above about 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, or greater than 900 nm.

### II. Process for Preparing Fluorescent Cyanine Dyes

The cyanine dyes may be prepared by methods known in the art including as described in the Examples (see Scheme 1 and Scheme 2) or as described in Zhegalova et al., Contrast Media Mol Imaging 2014; 9(5): 355-62, which is hereby incorporated by reference in its entirety. By way of non-limiting example, referring to Scheme 1, indole 3 was prepared via Fischer indole synthesis from 4-hydrazinylbenzenesulfonic acid (1) and 3-methylbutan-2-one (2). Known indolium salts 7 and 8 were prepared by alkylation of the corresponding indoles 4 and 6 with 1,3-propanesultone. Pre-activation of the Vilsmeier type reagent 9 with acetic anhydride was followed by addition of the indolium salt 7 (1.5:1 molar ratio) and acetic acid using standard procedures. After 4 h of stirring at reflux temperature, acetic acid was evaporated and the residue was washed with ethyl acetate several times to remove the unreacted reagent 9. Ethyl acetate was removed under vacuum and the intermediate 10 (hygroscopic) was immediately transferred to a vial, dissolved in acetic anhydride and pyridine (1:1) solvent ratio. Indolium salt 8 was added and the vial was heated to 110° C. for 10 min. The reaction was followed by the appearance and growth of an absorption peak at ca. 750 nm corresponding to the desired LS755 product and the vanishing of the 506 nm peak of the acetate form of the half-dye 10. Upon completion, the mixture was cooled, triturated with ethyl acetate, filtered, washed with ethyl acetate and 2-propanol, dried under reduced pressure and purified on a reverse phase column. The major product in the reaction mixture was found to be LS755 (>80 area-percent in LCMS). Detailed reaction schemes are delineated in the Examples.

### III. Conjugation of Fluorescent Cyanine Dyes to a Biomolecule

The fluorescent cyanine dyes may be attached to a biomolecule or a ligand to form a conjugated substrate. Attachment may be, for example, by covalent bonding, ionic bonding, dated bonding, hydrogen bonding, and other forms of molecular bonding.

Several types of biomolecules are suitable for conjugation to the cyanine dyes. For example, useful conjugated substrates of the invention include, but are not limited to, conjugates of antigens, small molecules, steroids, vitamins, drugs, haptens, metabolites, toxins, environmental pollutants, amino acids, peptides, proteins, photosensitizers, nucleotides, oligonucleotides, nucleic acids, carbohydrates, lipids, ion-complexing moieties, nanoparticles and non-biological polymers. In one exemplary embodiment, the conjugated substrate is a natural or synthetic amino acid; a natural or synthetic peptide or protein; or an ion-complexing moiety. Preferred peptides include, but are not limited to protease substrates, protein kinase substrates, phosphatase substrates, neuropeptides, cytokines, and toxins. Preferred protein conjugates include enzymes, antibodies, lectins, glycoproteins, histones, albumin, lipoproteins, avidin, streptavidins, protein A, protein G, casein, phycobiliproteins, other fluorescent proteins, hormones, toxins, growth factors, and the like. In another exemplary embodiment, the conjugated substrate is a nanoparticle.

The point of attachment of the biomolecule to the cyanine dye can and will vary depending upon the embodiment. In certain embodiments, the point of attachment may be at position R⁴ of any of the compounds described in Section (I) above. In certain embodiments, the point of attachment may be at position R⁵ of any of the compounds described in Section (I) above. In another embodiment, the point of attachment may be at position R³ of any of the compounds described in Section (I) above. In yet another embodiment, the point of attachment may be at position R¹² of any of the molecules described in Section (I) above. In still yet another embodiment, the point of attachment may be at position R¹ of any of the molecules described in Section (I) above. In a different embodiment, the point of attachment may be at position R⁹ of any of the molecules described in Section (I) above. In some embodiments, the point of attachment may be at any one of position R², R⁶, R⁷, R⁸, R¹⁰, or R¹² of any of the molecules described in Section (I) above. It is also envisioned that more than one biomolecule may be conjugated to the cyanine dye. For example, two, three or more than three biomolecules may be conjugated to the cyanine dye.

Several methods of linking dyes to various types of biomolecules are well known in the art. For example, methods for conjugating dyes to a biomolecule are described in R. Haughland, The Handbook A Guide to Fluorescent Probes and Labeling Technologies, 9^{th} Ed., 2002, Molecular Probes, Inc. and the references cited therein; and Brindley, 1992, Bioconjugate Chem. 3:2, which are all incorporated herein by reference. By way of example, a cyanine dye may be covalently attached to DNA or RNA via one or more purine or pyrimidine bases through an amide, ester, ether, or thioether bond; or is attached to the phosphate or carbohydrate by a bond that is an ester, thioester, amide, ether, or thioether. Alternatively, a cyanine dye may be bound to the nucleic acid by chemical post-modification, such as with platinum reagents, or using a photoactivatable molecule such as a conjugated psoralen. The Examples provided below provide methods for conjugating a dye to a protein or nanoparticle.

### IV. Uses of the Fluorescent Cyanine Dyes

The cyanine dyes of the invention are useful in many applications including those described for other cyanine dyes in U.S. Pat. Nos. 7,172,907; 5,268,486; and U.S. Patent Application Nos. 20040014981; and 20070042398, each of which is incorporated herein by reference. For example, fluorescent dyes may be used in imaging with techniques such as those based on fluorescence detection, including but not limited to fluorescence lifetime, anisotropy, photoinduced electron transfer, photobleaching recovery, and non-radioactive transfer. The fluorescent cyanine dyes, as such, may be utilized in all fluorescent-based imaging, microscopy, and spectroscopy techniques including variations on such. In addition, they could also be used for photodynamic therapy and in multimodal imaging. Exemplary fluorescence detection techniques include those that involve detecting fluorescence generated within a system. Such techniques include, but are not limited to, fluorescence microscopy, fluorescence activated cell sorting (FACS), fluorescent flow cytometry, fluorescence correlation spectroscopy (FCS), fluorescence lifetime imaging (FLIM), fluorescence in situ hybridization (FISH), multiphoton imaging, diffuse optical tomography, molecular imaging in cells and tissue, superresolution fluorescence imaging, fluorescence imaging with one nanometer accuracy (FIONA), free radical initiated peptide sequencing (FRIPs), and second harmonic retinal imaging of membrane potential (SHRIMP), as well as other methods known in the art.

Alternatively, the fluorescent cyanine dyes can be used as markers or tags to track dynamic behavior in living cells. In this regard, fluorescence recovery after photobleaching (FRAP) can be employed in combination with the subject fluorescent cyanine dyes to selectively destroy fluorescent molecules within a region of interest with a high-intensity laser, followed by monitoring the recovery of new fluorescent molecules into the bleached area over a period of time with low-intensity laser light. Variants of FRAP include, but are not limited to, polarizing FRAP (pFRAP), fluorescence loss in photo-bleaching (FLIP), and fluorescence localization after photobleaching (FLAP). The resulting information from FRAP and variants of FRAP can be used to determine kinetic properties, including the diffusion coefficient, mobile fraction, and transport rate of the fluorescently labeled molecules. Methods for such photo-bleaching based techniques are described in Braeckmans, K. et al., Biophysical Journal 85: 2240-2252, 2003; Braga, J. et al., Molecular Biology of the Cell 15: 4749-4760, 2004; Haraguchi, T., Cell Structure and Function 27: 333-334, 2002; Gordon, G. W. et al., Biophysical Journal 68: 766-778, 1995, which are all incorporated herein by reference in their entirety.

Other fluorescence imaging techniques are based on non-radioactive energy transfer reactions that are homogeneous luminescence assays of energy transfer between a donor and an acceptor. Such techniques that may employ the use of the subject fluorescent dyes include, but are not limited to, FRET, FET, FP, HTRF, BRET, FLIM, FLI, TR-FRET, FLIE, smFRET, and SHREK. These techniques are all variations of FRET.

### (a) In Vivo Techniques

In an aspect, an asymmetrically charged dye of the invention may be used for imaging in a subject. The method generally comprises administering to a subject an asymmetrically charged dye of the invention, wherein the dye is conjugated to a biomolecule, and detecting the asymmetrically charged dye in the subject.

A suitable subject includes a human, a livestock animal, a companion animal, a lab animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In certain aspects, a pharmacologically effective amount of an asymmetrically charged cyanine dye of the invention may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, intratracheal, intraneural in peripheral nerves, or suppository administration. Local administration, including directly into the central nervous system (CNS) includes but is not limited to via a lumbar, intraventricular or intraparenchymal catheter or using a surgically implanted controlled release formulation. In a specific embodiment, the dye is administered intratracheally.

The asymmetrically charged cyanine dye may be formulated into a composition suitable for administration to a subject. Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For imaging, a detectable amount of cyanine dye of the invention is administered to a subject. A “detectable amount”, as used herein to refer to a diagnostic composition, refers to a dose of such a composition that the presence of the composition can be determined in vivo or in vitro. A detectable amount will vary according to a variety of factors, including but not limited to chemical features of the biomolecule being labeled, the structural features of the dye, labeling methods, the method of imaging and parameters related thereto, metabolism of the conjugated dye in the subject, the stability of the dye (e.g. the half-life of a cyanine dye of the invention), the time elapsed following administration of the dye prior to imaging, the route of administration, and the physical condition and prior medical history of the subject. Thus, a detectable amount can vary and can be tailored to a particular application. After study of the present disclosure, and in particular the Examples, it is within the skill of one in the art to determine such a detectable amount.

The cyanine dye may be detected after administration to a subject. In an embodiment, the cyanine dye may be detected 5, 10, 15, 20, 25, 30, 35, 40, 45, 50, 55 or 60 min after administration. In another embodiment, the cyanine dye may be detected 1, 2, 3, 4, 5, 6, 7, 8, 9, 10, 11, 12, 13, 14, 15, 16, 17, 18, 19, 20, 21, 22, 23 or 24 hours after administration. In still another embodiment, the cyanine dye may be detected 1, 2, 3, 4, 5, 6, or 7 days after administration. In still yet another embodiment, the cyanine dye may be detected more than 7 days after administration.

Optical Imaging (OI) as known to those of skill in the art may be used to detect the cyanine dye in the subject. The whole subject may be imaged or a specific region of the subject may be imaged to detect the cyanine dye. If a specific region of the subject is imaged, the region may be chosen based on the biomolecule used, the route of administration, pharmacokinetics of the composition, and/or bioavailability of the composition. In an embodiment, a biomolecule is chosen that targets to a specific tissue such that that tissue may specifically be imaged. For example, an epitope binding agent or antibody targeting a specific tissue and/or tumor is conjugated to a dye of the invention such that that specific tissue and/or tumor may specifically be imaged. In such a manner, the image may be used to diagnose a disease or tumor and/or the image may be used to guide therapy to a disease or tumor. In another embodiment, the route of administration may cause concentration in a specific tissue such that that tissue may be imaged or treated. For example, a dye of the invention conjugated to a biomolecule may be administered intratumorally such that the tumor may be imaged. In such an embodiment, a dye of the invention may be conjugated to a therapeutic agent such that the tumor may be imaged and guide therapy to the tumor. In a specific embodiment, the route of administration is intratracheally such that the lung is imaged.

In another aspect, an asymmetrically charged cyanine dye of the invention may be used in a method for image guided drug delivery. Image-guided drug delivery can be used to non-invasively visualize and quantify probe accumulation at the target site, to validate (triggered) drug release at the target site, and to longitudinally monitor drug efficacy. Accordingly, an asymmetrically charged cyanine dye of the invention may be used to visualize and quantify the biodistribution and target site accumulation of drugs and drug delivery systems, and to non-invasively assess their efficacy.

### DEFINITIONS

The term “acyl,” as used herein alone or as part of another group, denotes the moiety formed by removal of the hydroxyl group from the group —COOH of an organic carboxylic acid, e.g., RC(O)—, wherein R is R′, R₁O—, R′R₂N—, or R₁S—, R₁ is hydrocarbyl, heterosubstituted hydrocarbyl, or heterocyclo and R₂ is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

As used herein, the term “functional group” includes a group of atoms within a molecule that is responsible for certain properties of the molecule and/or reactions in which it takes part. Non-limiting examples of functional groups include, alkyl, carboxyl, hydroxyl, amino, sulfonate, phosphate, phosphonate, thiol, alkyne, azide, halogen, and the like.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “linking group” includes a moiety on the compound that is capable of chemically reacting with a functional group on a different material (e.g., biomolecule) to form a linkage, such as a covalent linkage. See R. Haughland, The Handbook—A Guide to Fluorescent Probes and Labeling Technologies, 9^{th} Edition, Molecular probes, Inc. (1992). Typically, the linking group is an electrophile or nucleophile that can form a covalent linkage through exposure to the corresponding functional group that is a nucleophile or electrophile, respectively. Alternatively, the linking group is a photoactivatable group, and becomes chemically reactive only after illumination with light of an appropriate wavelength. Typically, the conjugation reaction between the dye bearing the linking group and the material to be conjugated with the dye results in one or more atoms of the linking group being incorporated into a new linkage attaching the dye to the conjugated material.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Asymmetrically Charged Fluorophore LS755

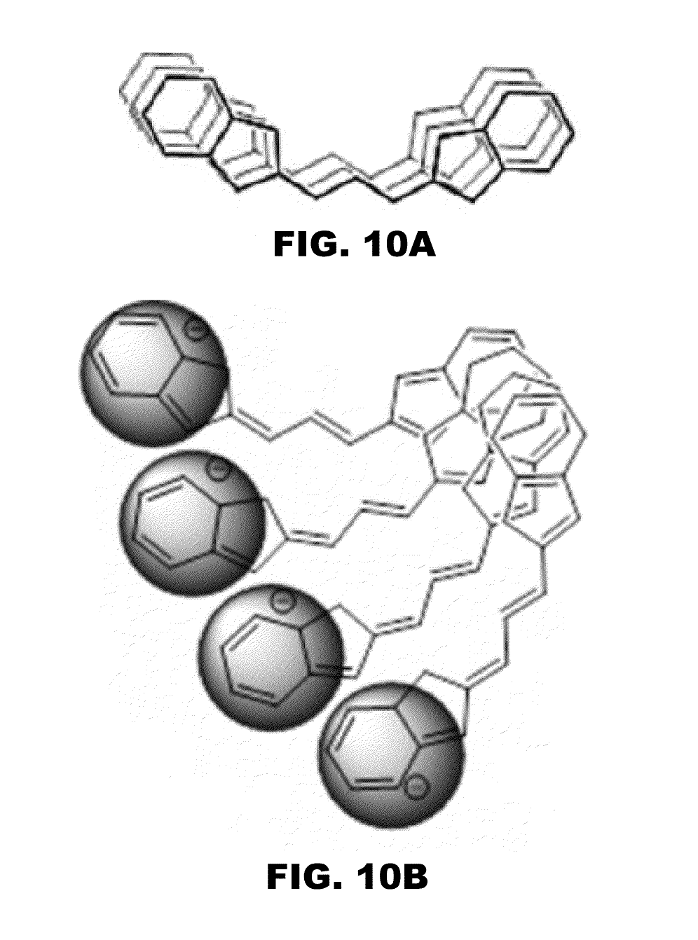

Herein, we optimized the structure of the labeling dye to minimize quenching. Our approach was to eliminate the self-aggregation of the dyes by increasing the asymmetry of the charge density on the chromophore. We hypothesized that the charge asymmetry would lead to repulsion of the fluorophores from each other in a twisted fashion similar to the geometry illustrated in FIG. 10. With such architecture of increased torsional angles, we expected to decrease the quenching of the dyes by breaking both strong and weak couplings between the individual dyes on the surface of the protein.

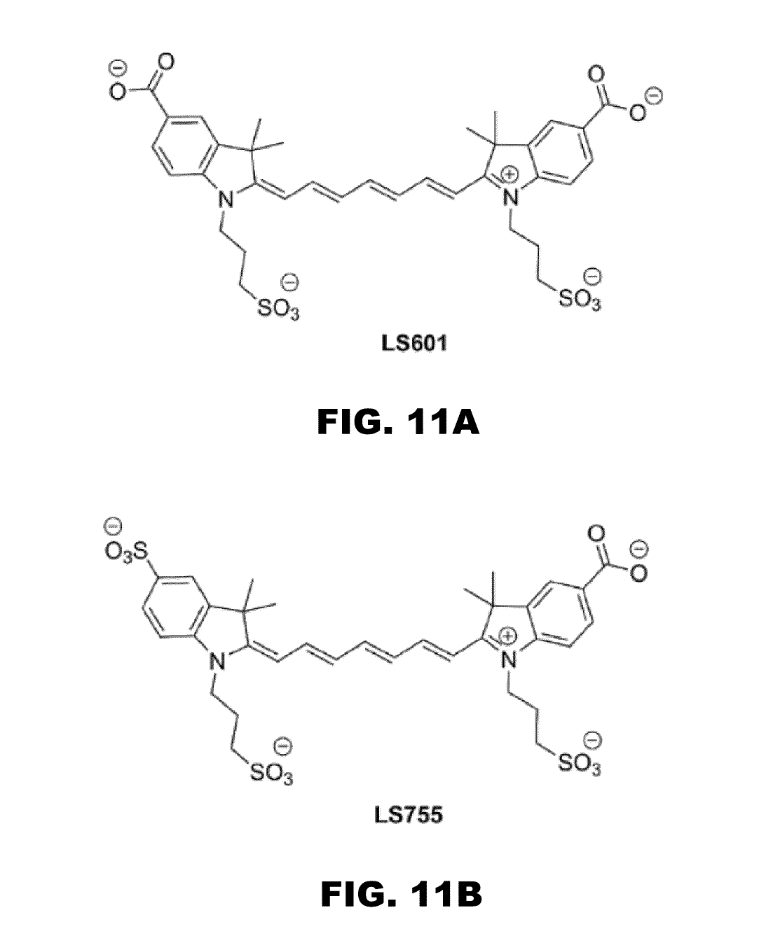

We prepared an asymmetrically charged fluorophore LS755 (FIG. 11) via the synthesis shown in Scheme 1. The dye was structurally similar to a previously published NIR dye LS601 that showed aggregation upon conjugation to macromolecules such as IgG. In LS755 one of the carboxylic groups is replaced with a sulfonate group. In a free nonconjugated form, both indoles from each dye carry charges. Upon conjugation, only one charge at the indole part remains.

Briefly, indole 3 was prepared via Fischer indole synthesis from 4-hydrazinylbenzenesulfonic acid (1) and 3-methylbutan-2-one (2). Known indolium salts 7 and 8 were prepared by alkylation of the corresponding indoles 4 and 6 with 1,3-propanesultone. Pre-activation of the Vilsmeier type reagent 9 with acetic anhydride was followed by addition of the indolium salt 7 (1.5:1 molar ratio) and acetic acid using standard procedures. After 4 h of stirring at reflux temperature, acetic acid was evaporated and the residue was washed with ethyl acetate several times to remove the unreacted reagent 9. Ethyl acetate was removed under vacuum and the intermediate 10 (hygroscopic) was immediately transferred to a vial, dissolved in acetic anhydride and pyridine (1:1) solvent ratio. Indolium salt 8 was added and the vial was heated to 110° C. for 10 min. The reaction was followed by the appearance and growth of an absorption peak at ca. 750 nm corresponding to the desired LS755 product and the vanishing of the 506 nm peak of the acetate form of the half-dye 10. Upon completion, the mixture was cooled, triturated with ethyl acetate, filtered, washed with ethyl acetate and 2-propanol, dried under reduced pressure and purified on a reverse phase column. The major product in the reaction mixture was found to be LS755 (>80 area-percent in LCMS).

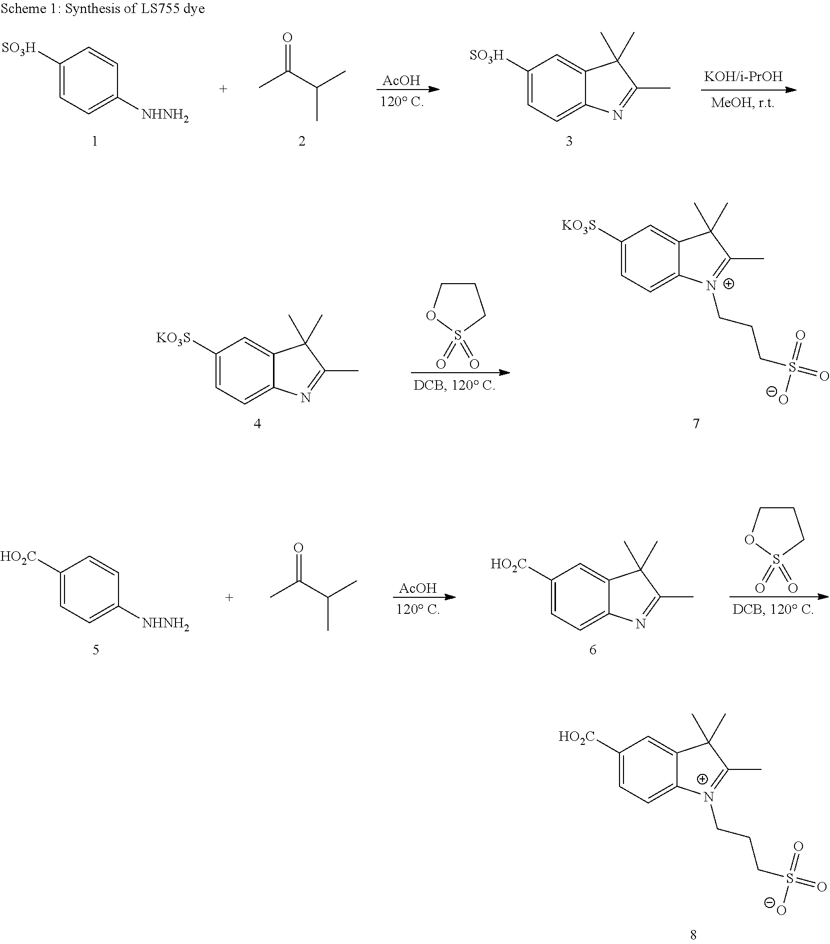

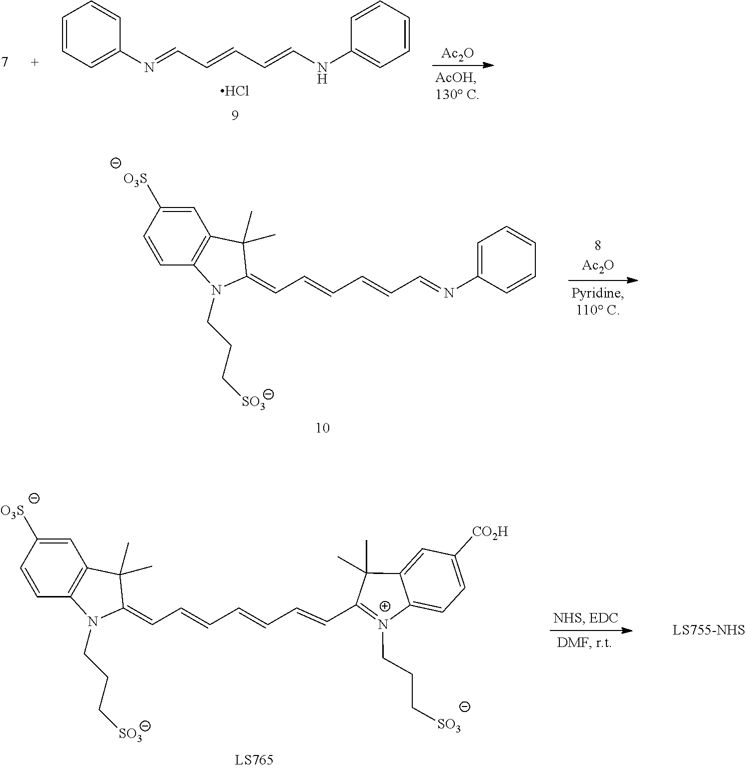

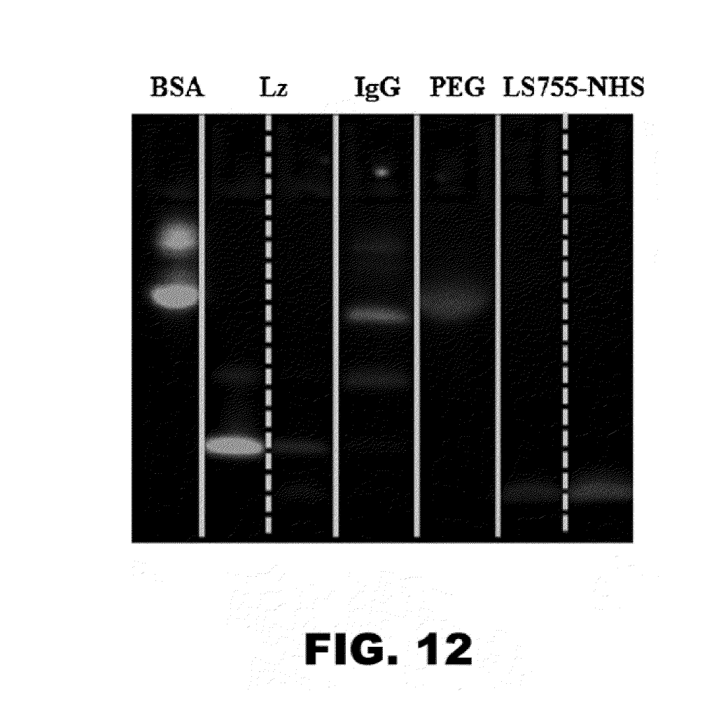

The conjugation of the dye to the amino groups on proteins occurred efficiently with high yield via conventional NHS chemistry as we described previously. For that, the carboxylic group of LS755 was converted into a corresponding NHS-ester in the presence of 1-ethyl-3-(3-dimethylaminopropyl) carbodiimide (EDC; Scheme 1). The pre-activated dye LS755-NHS in the lyophilized form was stable for at least several months. The coupling reactions to polypeptides were conducted in a bicarbonate buffer, and the products were purified using Sephadex columns. The conjugation of LS755 to the macromolecules and purity of conjugates were assessed using SDS-PAGE (FIG. 12). Single-chained proteins BSA and lysozyme showed bands corresponding to their molecular weights, and IgG composed of four peptide chains showed bands corresponding to their heavy and light chains. A conjugate LS755-PEG40 kDa was used as a molecular weight marker and showed a single spot on the gel. A free dye, LS755-NHS, appeared at the bottom of the gel near 1 kDa as expected. The purified conjugates showed no free dye.

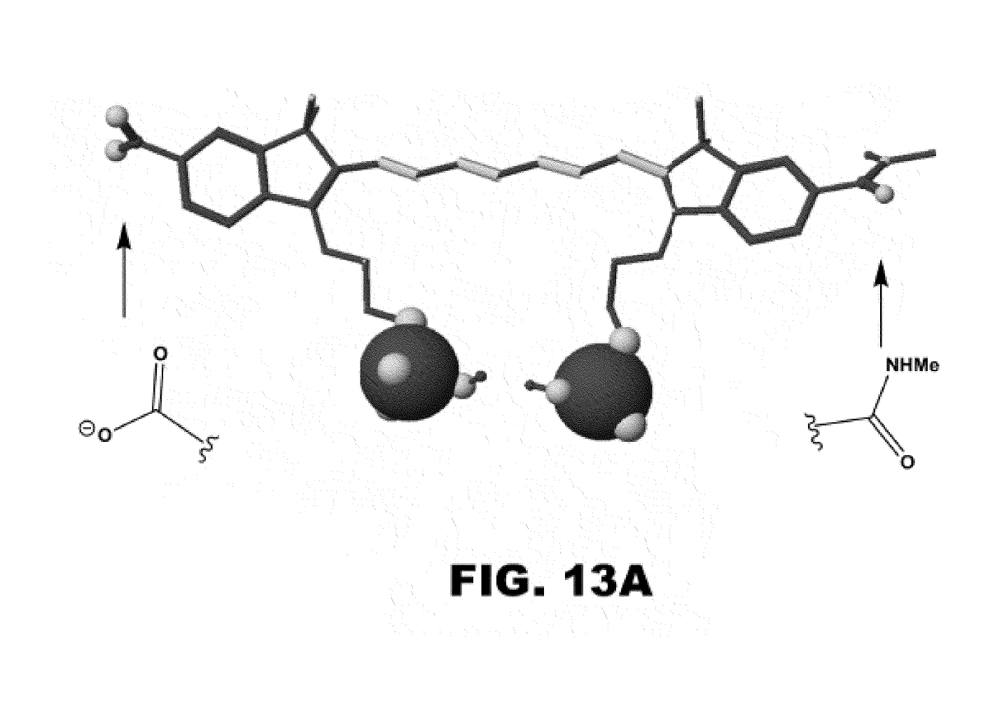

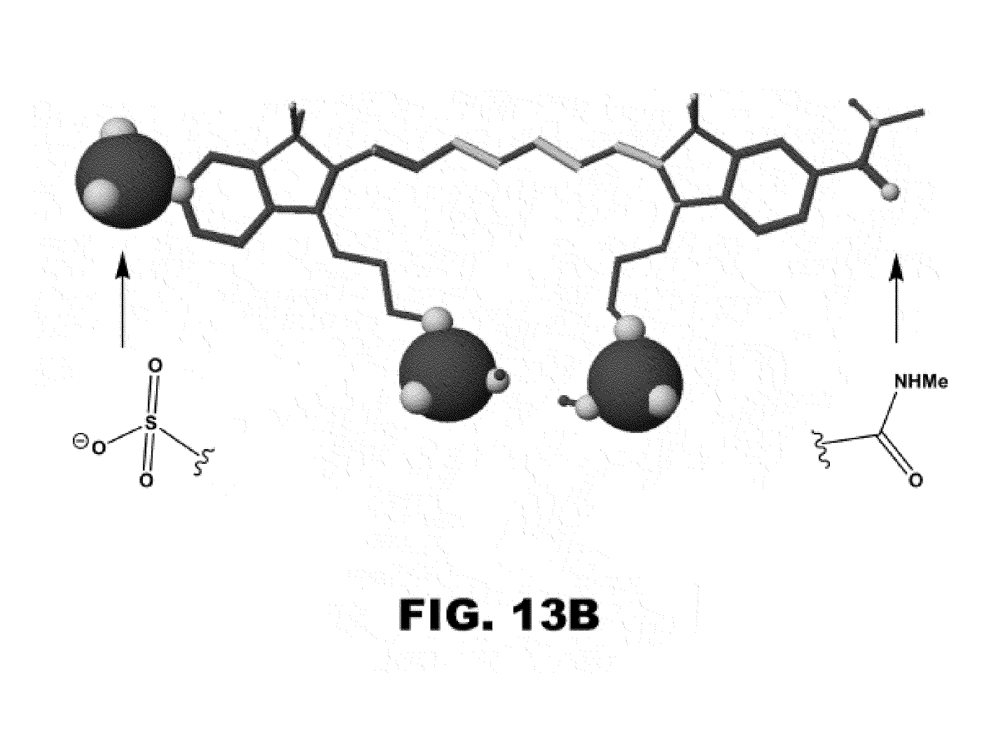

Upon conjugation, the dyes were expected to become nonsymmetrically charged under physiological conditions given that the values of pKa of carboxylic acid and sulfonate are in the range of pKa 3-4. Molecular modeling with MM3/PM5 parameters demonstrated that the charges are localized on the indole parts without delocalization across the conjugate system of the dyes (FIG. 13). As expected, the charge imposed by sulfonate-carrying indole (in LS755) was significantly higher than that in LS601, leading to a strong charge asymmetry of the dye in a dye conjugate system.

The replacement of a carboxylic group in LS601 with sulfonate (LS755) caused a 7-10 nm hypsochromic shift in absorption and emission spectra. Sulfonate is more electronegative and therefore is anticipated to cause a bathochromic shift opposite to what was expected. However, the results are in agreement with Kuhn's rationale for unsymmetrical dyes that predicts a blue shift in unsymmetrical polyenes.

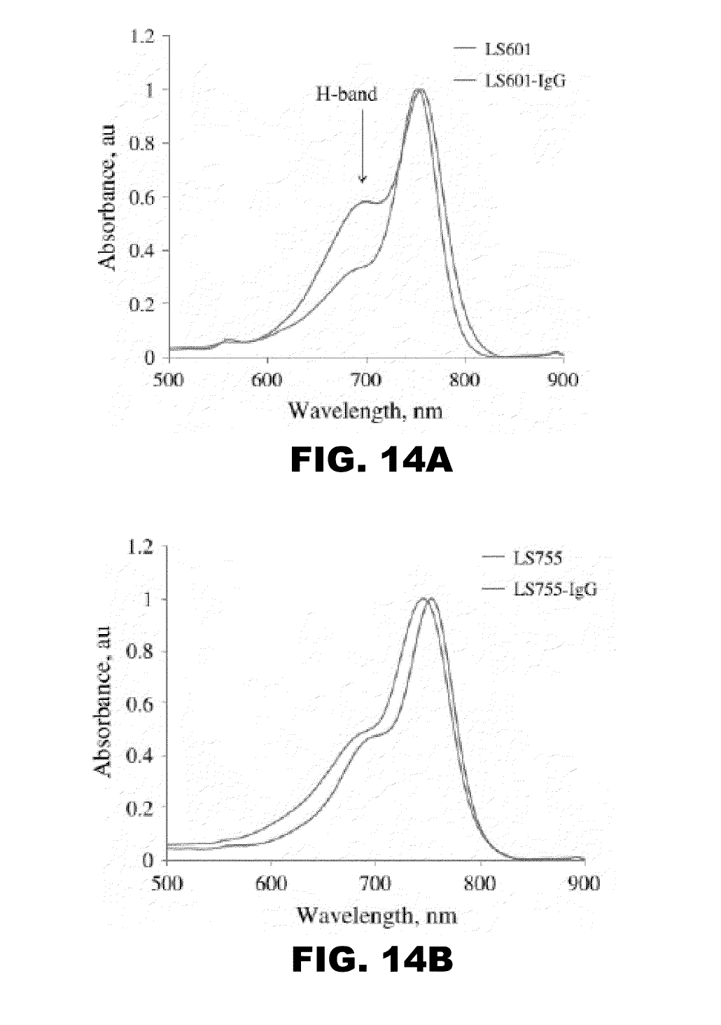

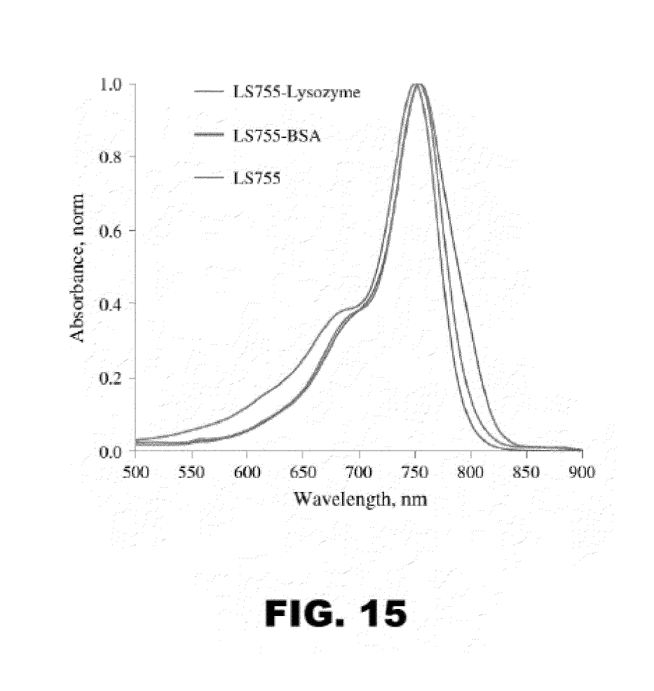

The most dramatic change between the dyes was observed in the shape of the absorption peaks of dye-peptide aggregates. The aggregation of dye molecules, such as cyanines, is commonly recognized as a hypsochromically shifted H-band in the absorption spectra of the conjugates, as shown in FIG. 14 for an LS601-IgG conjugate. It is generally accepted that H-aggregates are composed of dye molecules stacked in a plane-to-plane fashion. This type of aggregation leads to the undesirable loss of fluorescence. In contrast, the absorption spectra LS755-IgG demonstrated the disappearance of the H-band. Similar disappearance was also observed for other LS755 conjugates (FIG. 15).

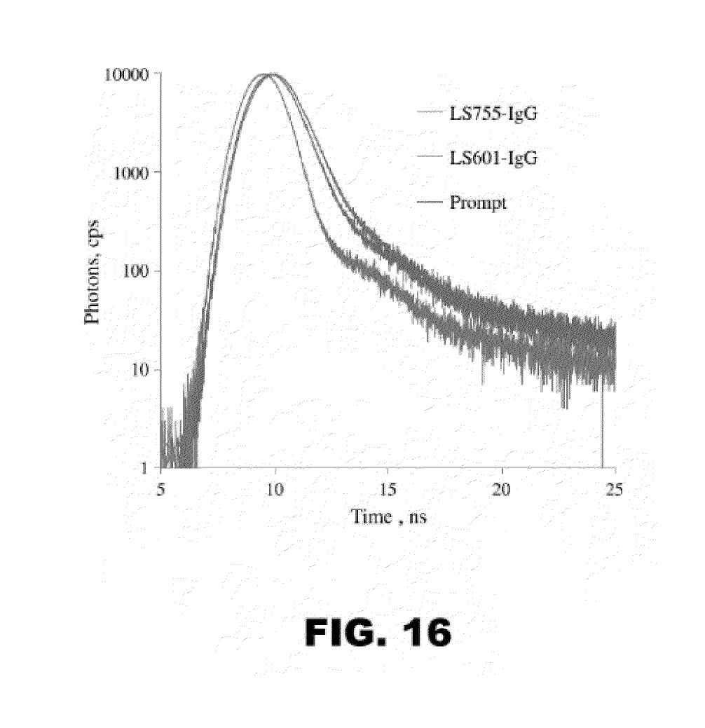

The fluorescence lifetime reflects the quenching of the probes, with shorter lifetimes indicating a higher degree of quenching. In solvents with strong solvation powers (DMSO) the fluorescence lifetime of LS755 (1.21 ns) was shorter than that of LS601 (1.29 ns). However, in solvents with weak solvation powers (water), that usually promotes aggregation of the cyanine dyes, the lifetime of LS755 was slightly greater (0.43 vs 0.42 ns for LS601 under the same conditions), reflecting a lower aggregation of LS755. The lifetime difference between the two dyes became even more apparent when measuring their conjugates to IgG (0.65 and 0.53 ns for LS755-IgG and LS601-IgG correspondingly; FIG. 16 and Table 1). The fluorescence lifetime of cyanines in dye-protein conjugates usually increases owing to a decrease in the rate of quenching caused by the solvent and the changes in the molecular environment (such as polarity) around the probe. Thus, the fluorescence lifetime of the dye conjugates is expected to be within two limits in DMSO and water.

In summary, we propose a new type of NIR fluorescent dye with an asymmetrical charge distribution to prevent an aggregation of the dye on the surface of a polypeptide with multiple sites of labeling. The first example of this type, a cyanine dye, LS755, was synthesized with a strong negatively charged sulfonate group on one side of the chromophore. Upon conjugation of LS755 to macromolecules such as antibodies, proteins and enzymes, a negligible formation of the H-band with high quantum yield and long fluorescence lifetime was observed. This method of labeling opens a pathway to more efficient fluorescent targeting probes with minimum dye aggregation. The latter leads to a smaller amount of the probe and therefore to a safer imaging procedure. Future directions include optimizing the conjugation chemistry to preserve the high specificity of biological polypeptides to molecular targets.

TABLE 1
Optical probes of LS755 and dye-conjugates.

| | MW, | λ_{abs} | λₑₘ | τ | | |
|---|---|---|---|---|---|---|
| Probe | kDa | (nm) | (nm) | (ns) | Φ_{fl} | r |
| LS755 | 0.7 | 758^{a}/745^{c} | 790^{a} | 1.21^{a}/0.43^{c} | 0.260^{a}/0.077^{c} | 0.155 ± 0.001^{c} |
| LS755-IgG | 150 | 755^{c} | 773^{c} | 0.65^{b} | 0.097^{c} | 0.239 ± 0.002^{c} |
| LS755-BSA | 67 | 755^{c} | 764^{c} | 0.96^{b} | 0.131^{c} | 0.201 ± 0.001^{c} |
| LS755-Lz | 15 | 754^{c} | 770^{c} | 0.73^{b} | 0.131^{c} | 0.222 ± 0.002^{c} |
| LS601(8) | 0.7 | 769^{a}/752^{c} | 800^{a} | 1.29^{a}/0.42^{c} | 0.200^{a} | 0.244 ± 0.002^{c} |
| LS601-IgG(8) | 150 | 757^{c} | 773^{c} | 0.53^{c} | 0.078^{c} | 0.311 ± 0.008 |

^{a}DMSO;
^{b}20% serum in water;
^{c}water.
MW, Approximate molecular weight of the conjugate; λ_{abs}, absorption maximum; λₑₘ, emission maximum, Φ_{fl}, relative fluorescence quantum yield using a reference ICG in DMSO = 0.12 (32) [for using another value = 0.22 (33, 34), the quantum yield has to be adjusted accordingly]; r, fluorescence anisotropy.

### Example 2

### Conjugation of LS755 to Proteins

BSA-LS755 Conjugate.

BSA (10.8 mg) was dissolved in 400 μl of 0.1 M NaHCO₃ buffer and mixed with a solution of LS755-NHS (0.6 mg) in 50 μl DMSO. The reaction mixture was left shaking at room temperature for 3 h. The conjugate was purified on a Sephadex G-25 column with 1×PBS buffer. Fractions were evaluated using SDS-PAGE gel; those containing the product were collected and lyophilized.

Lysoszyme-LS755 conjugate.

Lysozyme (1.17 mg) from chicken egg white was dissolved in 400 μl of 0.1 M NaHCO₃ buffer and mixed with a solution of LS755-NHS (0.6 mg) in 50 μl DMSO. The reaction mixture was left shaking at room temperature for 3 h. The conjugate was purified on a Sephadex G-25 column with 1×PBS buffer. Fractions were evaluated using SDS-PAGE gel; those containing the product were collected and lyophilized.

IgG-LS755 Conjugate.

IgG from rat serum (reagent grade>95% by SDS-PAGE; 1 mg) was dissolved in 400 μl of 0.1 M NaHCO₃ buffer. LS755-NHS (17 μl) in 1.4 μl DMSO was added to this mixture and was shaken for 3 h at room temperature. The reaction mixture was purified on a Sephadex G-25 column with 1×PBS buffer. Fractions were evaluated using SDS-PAGE; those containing the product were collected and lyophilized.

### Example 3

### Conjugation of LS755 to Nanoparticles Carrying Amines Lead to Highly Fluorescent Nanoparticles

Gene therapy possesses potential for the treatment of various lung-related diseases. One of the barriers to successful gene therapy is uncontrolled delivery of therapeutic agents into the target cells. To address this problem, we developed a dual function near-infrared (NIR) labeled polymeric nanocarrier for optically monitoring effective site-specific delivery. The NIR nanoparticles were composed from a biodegradable cationic polymer PLA, covalently linked to a new type of NIR probe LS755 with antiquenching properties and electrostatically bound to a luciferase plasmid payload. The particles had high fluorescence brightness, low cell toxicity and successfully transfected cells with luciferase in vitro. The nanoconstructs were intratracheally delivered to lungs of mice and demonstrated strong fluorescent signal and stability in vitro allowing non-invasive visualization in vivo. Nanoparticles were shown to deliver the plasmid cargo to lung tissue and tracked during clearance from the lungs over a two-week period. This design of NIR nanoparticles demonstrated utility as a scaffold for image-guided delivery of gene therapeutic agents.

General Procedure for Conjugation and Purification of Asymmetric Dyes to Nanoparticles:

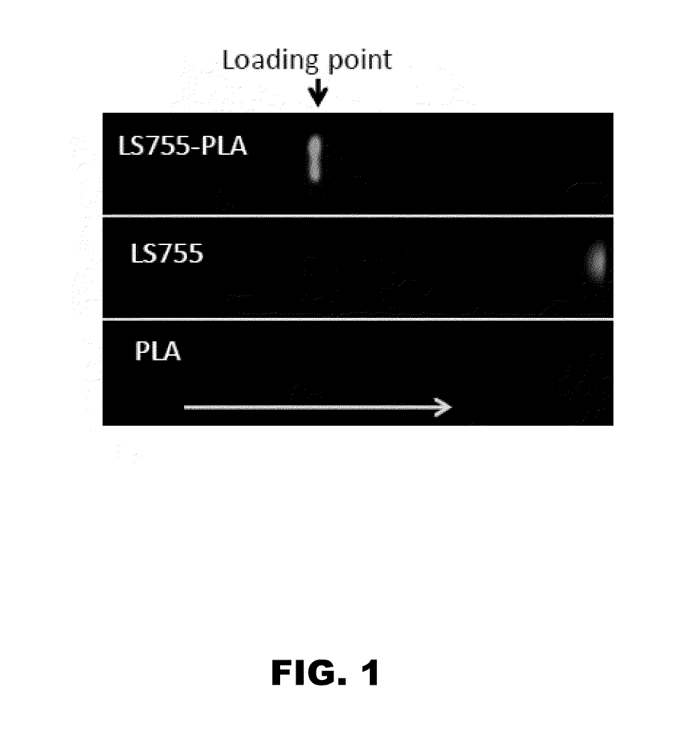

Amine carrying nanoparticles are dissolved in 0.1 M sodium bicarbonate buffer vortexing them for 30 sec. An appropriate NIR dye in its NHS activated form in DMSO solution was added in concentrations ranging from 0.05 to 0.5% in regards to concentration of amines on the nanoparticle. The reaction mixture was then placed in the shaker for 3 hours. Labeling of the nanoparticles with NIR dye was analyzed using gel electrophoresis using an agarose type 1 gel (FIG. 1) or precast Bio-Rad Any kD Mini-PROTEAN® TGX™ Gel. Free (non-conjugated) NIR dye was used as a control in all cases. Gel electrophoresis conditions: 150V, 30 min. Relatively large nanoparticles (˜25-200 nm) remained in the loading well due to their large size, a free dye traveled to the end of the gel due to its molecular weight (˜800 Da). Fluorescent nanoparticles can be optionally purified using electrodialysis.

### Example 4

### Fluorescence Spectroscopy Demonstrated High Emission and Quantum Yield of the LS755-NP Conjugates

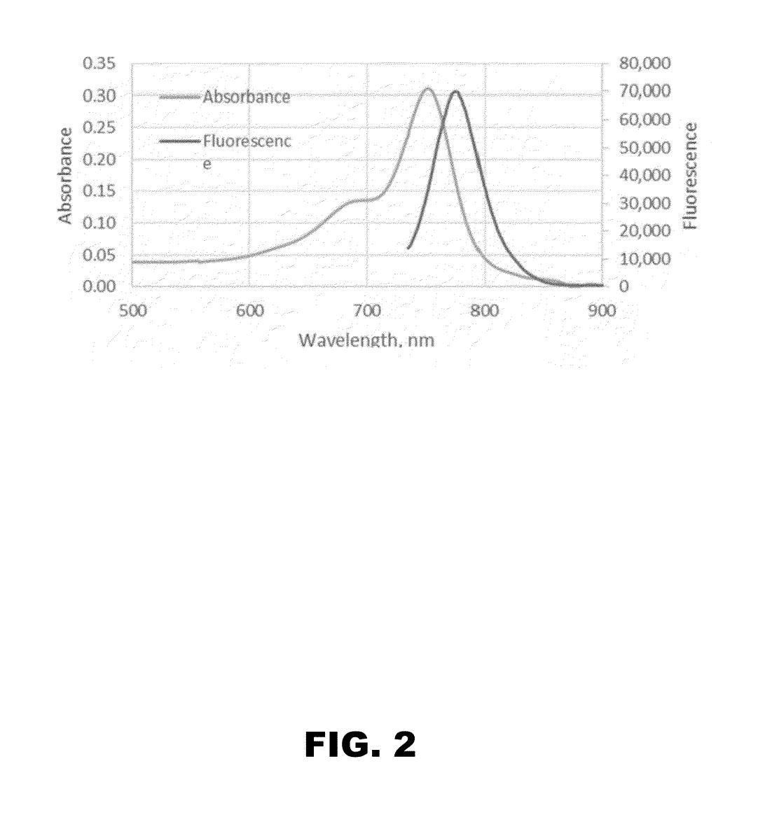

The compound was dissolved in 1% BSA/water to mimic biological conditions such as blood. Absorption spectra (FIG. 2) showed no H-type aggregation of the dye on the surface of the nanoparticle. Emission study showed high quantum yield of the nanoparticle of 10-34% depending on the loading (relative to ICG with QY=23%). LS755 works for labeling better (brighter, less aggregation) than either nonsymmetrical LS601 or LS634. Non-NHS LS755 doesn't stick to the nanoparticles.

### Example 5

### RAW Cells with LS755-NP Conjugates Demonstrate Potential of the Dye in Cell Imaging

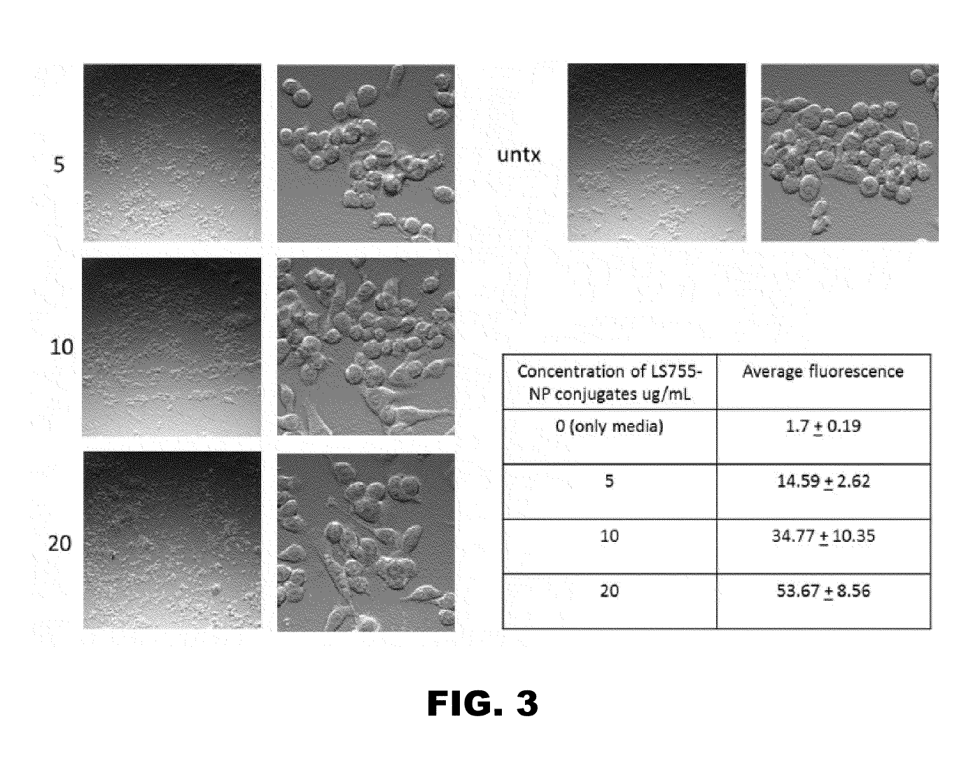

MatTek 35 mm glass-bottom-dishes with 2.5×10⁴ cells/well and LabTek 8-well chamber slides with 3.2×10⁴ cells/well were imaged with a microscope Olympus BX51 using Cy7 filtercube. Cells were treated with 5, 10, or 20 ug/ml of LS755-NP conjugates or left non-treated (media only) for 24 hours. FIG. 3 and Table 2 demonstrate that nanoparticles labeled with LS755 penetrate cells retain brightness in the cells and suitable for cell imaging.

TABLE 2
Average fluorescence of LS755-NP conjugates

| Concentration of LS755-NP conjugates | |
|---|---|
| μg/mL | Average fluorescence |
| 0 (only media) | 1.7 ± 0.19 |
| 5 | 14.59 ± 2.62 |
| 10 | 34.77 ± 10.35 |
| 20 | 53.67 ± 8.56 |

### Example 6

### Dye-NPs Conjugate have Low Cell Toxicity

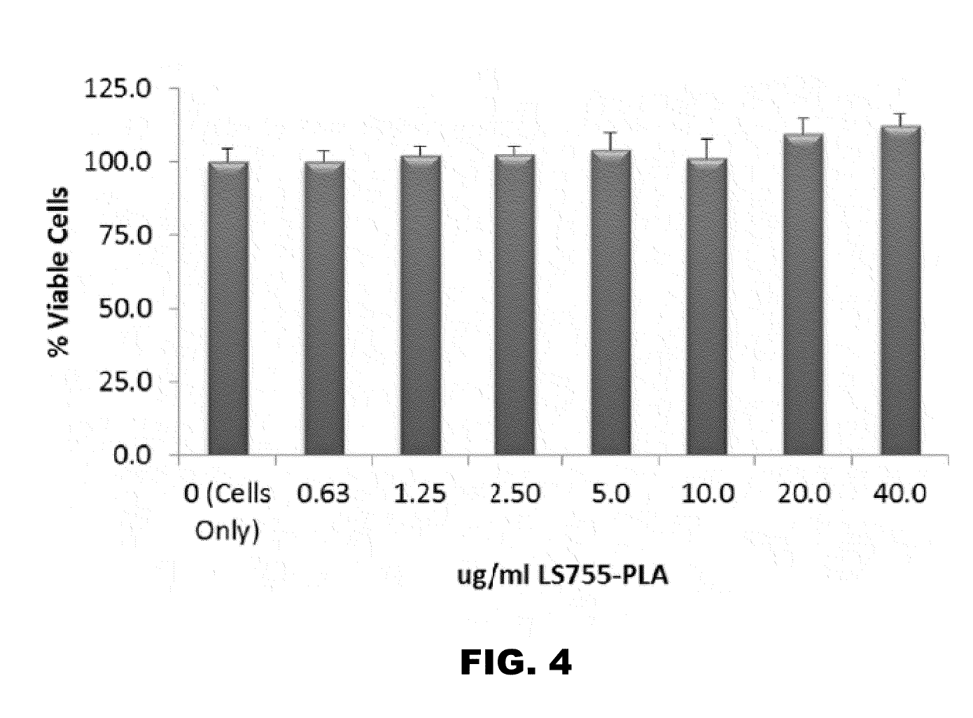

5×10⁴ RAW 264.7 cells/well were seeded in a 96-well plate and then immediately treated with dilutions of LS755-NPs in cell growth media. The cells+conjugates were then incubated at 37° C., 5% CO₂ for 24 hours at which-point the CellTiter-Glo Luminescent Cell Viability Assay (Promega) was performed. The percent cell viability was calculated relative to non-treated, media only control. FIG. 4 shows high viability of the LS755-NPs in RAW cells.

### Example 7

### Dye-NPs Conjugates have Low In Vivo Toxicity

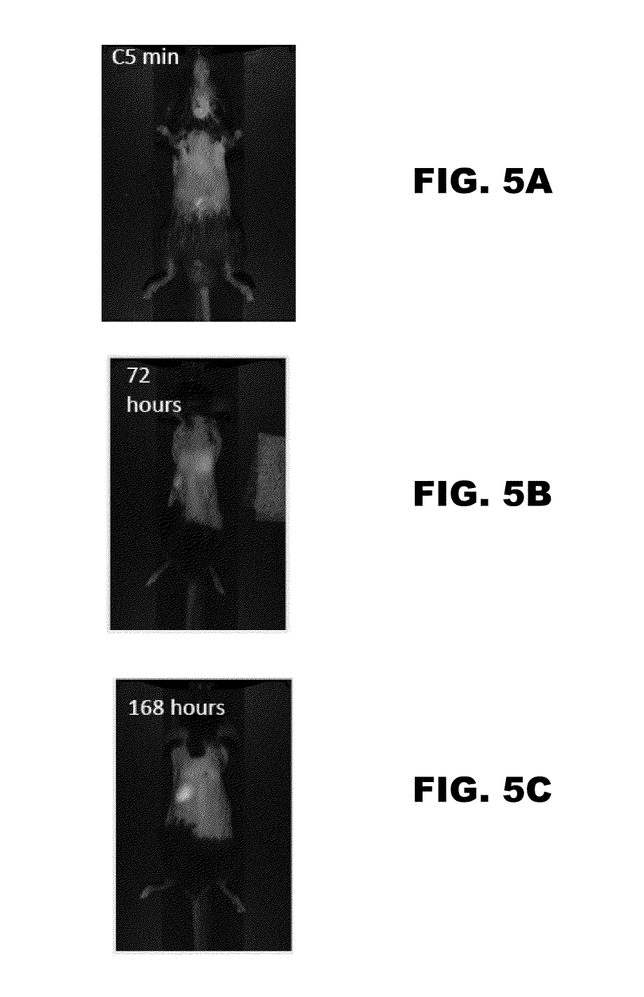

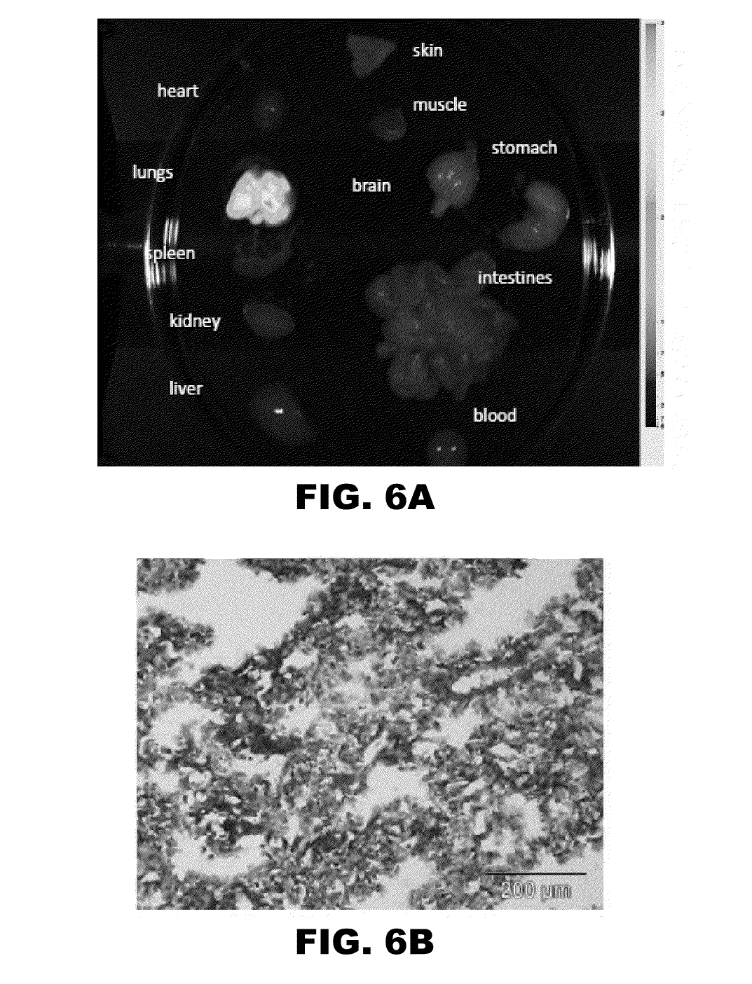

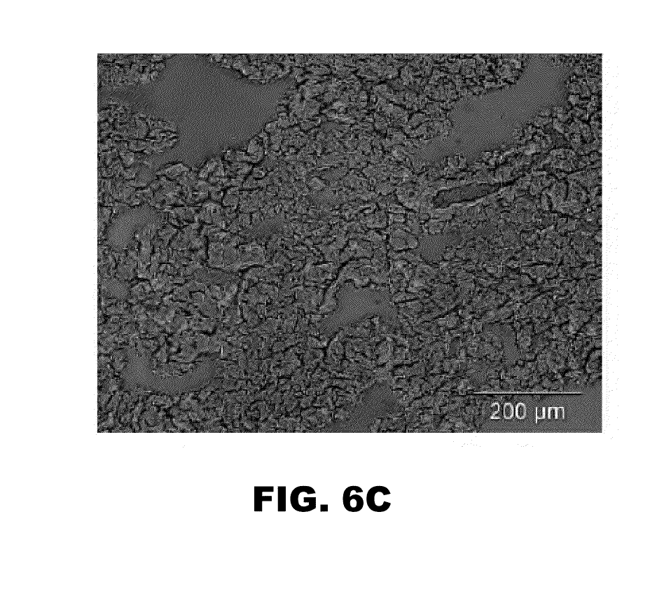

LS755-NPs conjugates were administered to mice intratracheally to visualize the distribution and stability of fluorescently labeled nanoparticles in vivo. After 168 hours the mice were in good shape. High fluorescence retained in lungs that was confirmed via biodistribution study (FIG. 5) and histological analysis of lung tissue thin slices (FIG. 6).

In conclusion, LS755 provides an excellent dye for labeling nanoparticles. The dye-NPs conjugates showed low cytotoxicity, and remained bright in vivo a week after delivery in lungs. These imaging nanoparticles could be used for image guided delivery of drugs or other diagnostic agents.

### Example 8

### Synthesis of LS822 (Asymmetric Fluorescent Dye with Abs/Em 650/670 Nm)

### 2,3,3-Trimethyl-3H-indole-5-sulfonic acid 3

The compound 3 (Scheme 2) was synthesized by conventional Fisher indole synthesis (IIIy and Funderburk 1968; Mujumdar, Ernst et al. 1993). Briefly, to a reacti-vial equipped with stir bar, acetic acid (3 mL), 3-methyl-2-butanone 2 (1.68 mL, 0.016 mol), and p-hydrazinobenzenesulfonic acid 1 (1 g, 0.0053 mol) were added. The mixture was heated to reflux and the reaction was monitored by TLC. R_{f}=0.67 (Silica, DCM-MeOH 3:1) The mixture was cooled to room temperature and then ether was added slowly until a pink solid separated. The precipitate collected by filtration and washed with ether (1.19 g, yield 94%). ¹H NMR (400 MHz, MeOD) δ=7.79 (d, 1H, J=2.0 Hz, aromatic 4-H), 7.15 (dd, 1H, J=8.0 Hz, 2.0 Hz, aromatic 6-H), 7.41 (d, 1H, J=8.0 Hz, aromatic 7-H), 1.31 (s, 6H, C(CH₃)₂). Singlet for 2-methyl did not appear in MeOD.

### 2,3,3-Trimethyl-3H-indole-5-sulfonate 4

This intermediate was synthesized according to standard procedures (Oushiki, Kojima et al. 2010). Briefly, product 3 (1.6 g, 6.7 mmol) was converted to the potassium salt 4 (Scheme 2) by stirring its solution in methanol (1.8 mL) with a saturated solution of potassium hydroxide in 2-propanol (26 mL). The resulting yellow precipitate was collected by filtration and washed with ether to give 4 as an orange solid. R_{f}=0.5 (Silica, DCM-MeOH 3:1). The filtrate was vacuum dried to afford 4. Compound 4 was used without further purification (1.19 g, yield 64%). ¹H NMR (400 MHz, MeOD) δ=7.79 (d, 1H, J=1.6 Hz, aromatic 4-H), 7.77 (dd, 1H, J=8.0 Hz, 2.0 Hz, aromatic 6-H), 7.54 (d, 1H, J=8 Hz, aromatic 7-H), 1.31 (s, 6H, C(CH₃)₂). Singlet for 2-methyl did not appear in MeOD.

### 2,3,3-Trimethyl-3H-indole-5-carboxylic acid 6

The compound 6 (Scheme 2) was synthesized by conventional Fisher indole synthesis (IIIy and Funderburk 1968; Mujumdar, Ernst et al. 1993). Briefly, to a reacti-vial equipped with stir bar, acetic acid (10 mL), 3-methyl-2-butanone (5.5 mL, 52 mmol), and 4-hydrazinobenzoic acid (3.33 g, 21.88 mmol) were added. The mixture was stir at room temperature for 10 min and then heated to reflux. The reaction was monitored by TLC. R_{f}=0.95 (Silica, DCM-MeOH 3:1) The mixture was cooled to room temperature and the solvent was evaporated off under reduced pressure and to it a saturated aqueous solution of NaHCO₃ (30 mL) was added and washed with DCM. The pH of the aqueous solution was adjusted to ˜2 with 2 M HCl and then extracted with DCM. The combined organic solution was then dried with Na₂SO₄, filtered and concentrated to dryness under reduced pressure to afford the product as brown solid (2.06 g, yield 46%). ¹H NMR (400 MHz, CDCl₃) δ=8.15 (d, 1H, J=8.0 Hz, aromatic 4-H), 8.07 (s, 1H, aromatic 6-H), 7.69 (d, 1H, J=8.0 Hz, aromatic 7-H), 2.40 (s, 3H, C(CH₃)), 1.30 (s, 6H, C(CH₃)₂).

### 2,3,3-Trimethyl-1-(3-sulfonatopropyl)-3H-indolinium-5-sulfonate 7

The compound 7 (Scheme 2) was synthesized according to the well-established literature procedure (Mujumdar, Ernst et al. 1993; Toutchkine, Nalbant et al. 2002). To a reacti-vial equipped with stir bar, the potassium salt 4 (0.745 g, 0.003 mol) and 1,3-propane sultone (0.4 g, 0.0033 mol) were mixed in 5 mL dichlorobenzene and heated at 120° C. for 12 h under nitrogen. The mixture was cooled to room temperature, then dichlorobenzene was decanted and the solid was triturated with ether to give product as quaternary salt (0.53 g, yield 43.8%). R_{f}=0.93 (C18, H₂O-MeOH 1:1). ¹H NMR (400 MHz, DMSO-d₆) δ=8.01-7.80 (m, 3H, aromatic ring), 4.63 (t, 2H, J=8 Hz), 2.82 (s, 3H, CH₃), 2.62 (t, 2H, J=6.4 Hz, CH₂SO₃), 2.14 (sex, 2H, J=8 Hz, CH₂CH₂CH₂), 1.54 (s, 6H, 2×CH₃).

### 2,3,3-Trimethyl-1-(3-sulfonatopropyl)-3H-indolinium-5-carboxylic acid 8

The compound 8 (Scheme 2) was synthesized according to the well-established literature procedure (Pham, Medarova et al. 2005). To a reacti-vial equipped with stir bar, 2,3,3-Trimethyl-3H-indole-5-carboxylic acid 6 (0.5 g, 0.0025 mol) and 1,3-propane sultone (0.458 g, 0.0038 mol) were mixed in 4 mL dichlorobenzene and heated at 120° C. for 24 h under nitrogen. The mixture was cooled to room temperature, then ether was added and the reaction mixture was filtered and washed with acetone. The residue was recrystallized with MeOH and acetone for 2 times and then was vacuum dried to afford brown solid 8 (0.64 g, yield 78.6%). R=0.8 (C18, H₂O-MeOH 1:1). ¹H NMR (400 MHz, DMSO-d₆) δ=8.37-8.16 (m, 3H, aromatic ring), 4.68 (m, 2H), 2.88 (s, 3H, CH₃), 2.63 (m, 2H, CH₂SO₃), 2.16 (m, 2H, CH₂CH₂CH₂), 1.57 (s, 6H, 2×CH₃).

LS822:

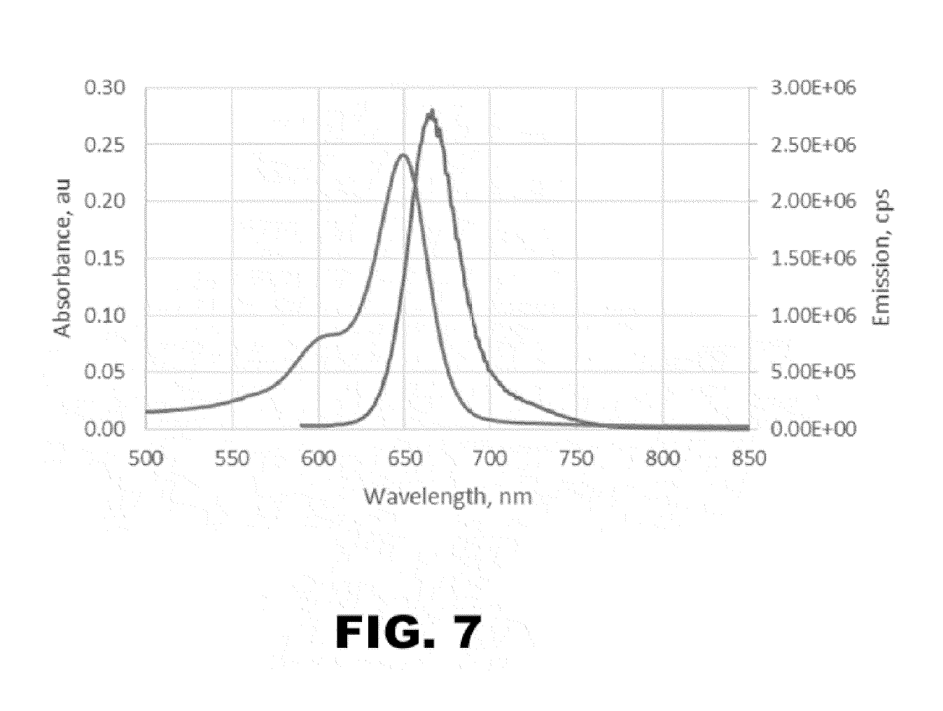

The dye LS822 was synthesized according to the procedure based on our previously publication procedure for LS755. Briefly, to a reacti-vial equipped with stir bar, indolenine 7 (213.34 mg, 0.534 mmol) and malonaldehyde bis(phenylimine) monohydrochloride (207.30 mg, 0.801 mmol) in a mixture of Ac₂O (3.2 mL) and AcOH (3.2 mL) was heated to reflux for around 4 hours. The progress of the reaction was monitored by both TLC(R_{f}=0.83 for 10, C18, H₂O-MeOH 1:1) and UV-Vis (disappearance of the absorption peak at 385 nm and the appearance of a strong peak at 456 nm). Acetic acid was removed with a rotary evaporator and the product was triturated by EtOAC to remove excess aniline 9. The crude intermediate 10 obtained was used in the next step without further purification. The intermediate 10 (171.12 mg, 0.35 mmol) was dissolved in an acetic anhydride (3.35 mL) and pyridine (3.35 mL) mixture. The UV spectrum shows an absorbance at 454 nm for the acetate form of the intermediate. Indolenine 8 was added and the reaction mixture was heated at 110° C. for 10 minutes and the reaction mixture turns to green (disappearance of the absorption peak at 454 nm and the appearance of a strong peak at 651 nm) (FIG. 7). The mixture was cooled to room temperature and then EtOAc was added to precipitate the dye. The gummy residue was washed with EtOAc and 2-propanol. Then the product was dried under reduced pressure to afford LS822 as blue solid (192.44 mg, yield 76%) R_{f}=0.75 (C18, H₂O-MeOH 1:1). ESI-MS m/z: 723.83 [M⁺].

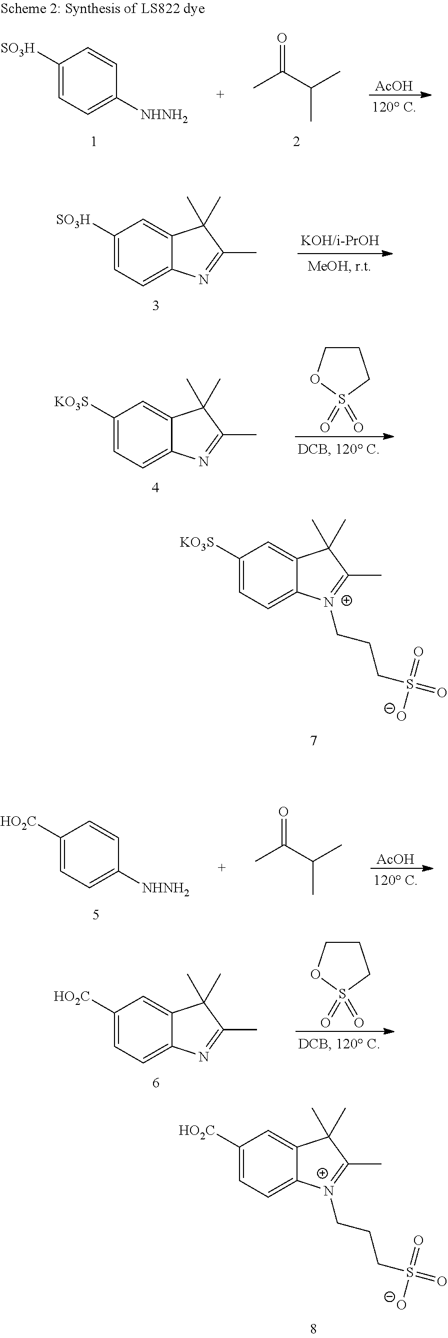

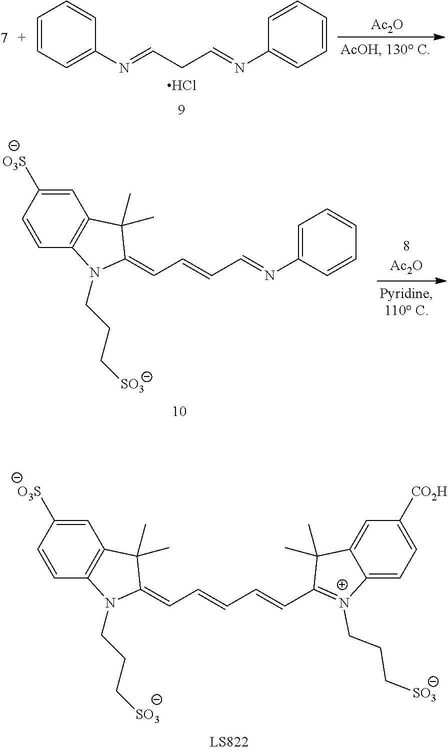

### Example 9

### Synthesis of LS822-NHS

LS822-NHS:

LS822-NHS-ester was synthesized according to the literature procedure (Mujumdar, Ernst et al. 1993; Markova, Fedyunyayeva et al. 2013) (Scheme 3). Briefly, to a reacti-vial equipped with stir bar, LS822 (48.95 mg, 0.0679 mmol) and was dissolved in dry DMF (2 mL), N-Hydroxysuccinimide (NHS) (23.41 mg, 0.2037 mmol) and 1-Ethyl-3-(3-dimethylaminopropyl)-carbodiimide (EDC) (39.01 mg, 0.2037 mmol) was added and the reaction mixture was stirred overnight at room temperature. The reaction was monitored by TLC for the production of LS822-NHS. R_{f}=0.25 (C18, H₂O-ACN 75:25). Diethyl ether was added to the reaction mixture to precipitate the product. The obtained product was redissolved in a minimal amount of methanol and triturated with diethyl ether. This step was repeated three times to give the desired NHS-ester as a blue solid (40 mg, yield 72%). ESI-MS m/z 820.93 [M⁺].

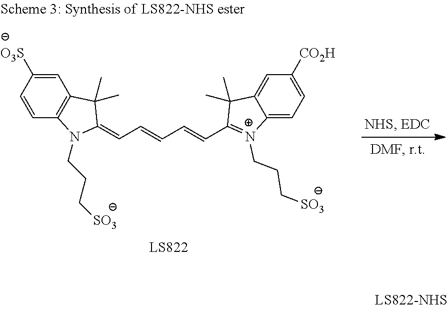

### Example 10

### Comparing the Brightness of the LS822 Upon Conjugation with the Brightness of Commercial Dyes with Flow Cytometry

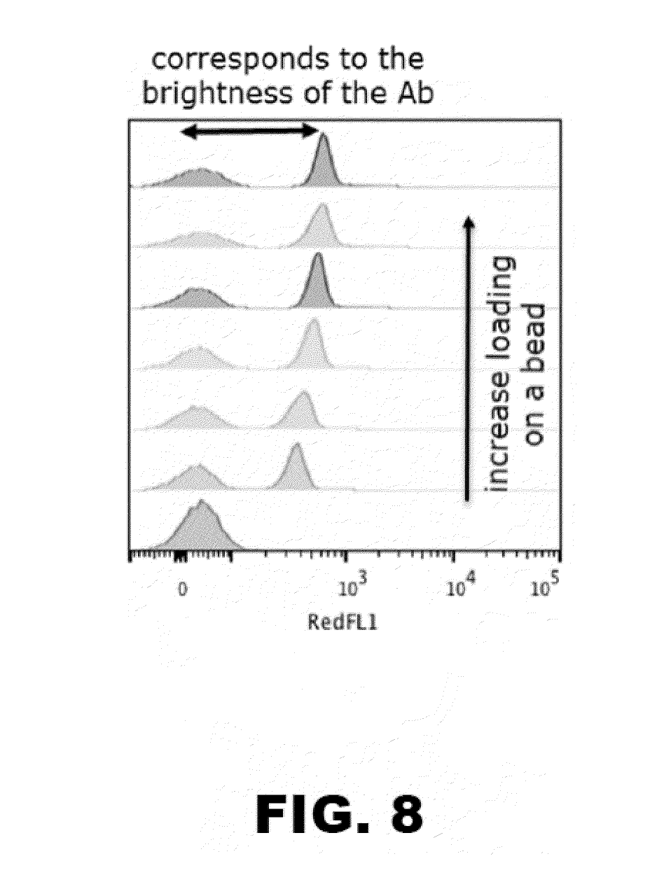

We developed a quantitative method to measure the brightness of fluorescently labeled antibodies. The method is based on flow cytometry with compensation beads. Compensation beads are beads of several micro diameter coupled to an antibody (positive beads) specific for the kappa light chain of immunoglobulin (Ig) from mouse, rat, or rat/hamster. When mix them with any kappa bearing fluorochrome-conjugated antibody makes the beads fluorescent. The level of fluorescence, as measured by flow cytometry, is linearly proportional to the brightness of the fluorescent antibody. Compensation beads set also contains a negative control, with no binding capacity, which consists of particles labeled with BSA or FBS. The ratio of these negative beads to positive beads is ˜1:1. Typical results with fluorescently labeled antibodies are shown in FIG. 8.

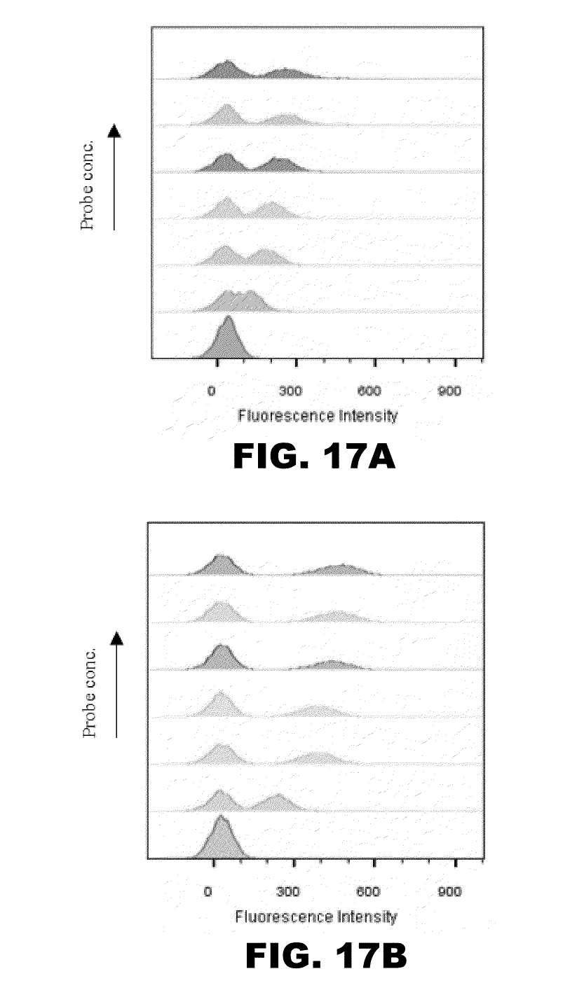

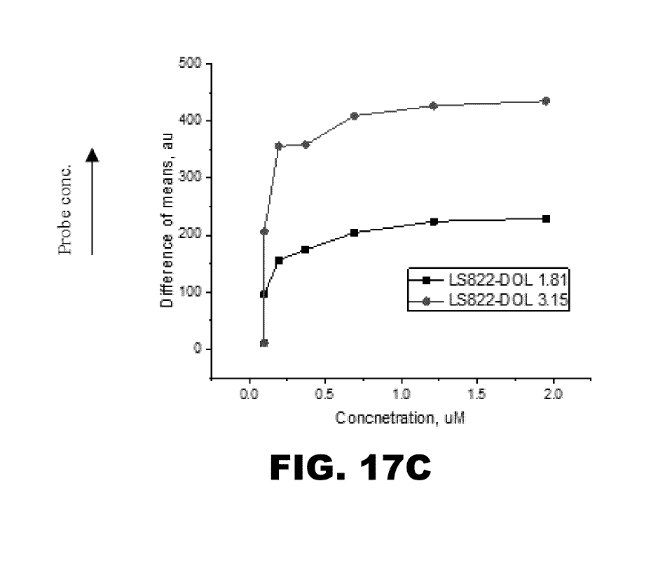

Using LS822 we tested whether the degree of labeling (DOL (corresponds to the number of dye molecules per one molecule of the antibody) affects the brightness of the labeled antibody. The higher degree of labeled antibody with a DOL 3.51 (FIG. 17A) vs 1.81 (FIG. 17B), showed proportionately higher intensity values at equal concentrations of conjugate (as shown in FIG. 17C), suggesting minimal to weak self-quenching of the dye after labeling.

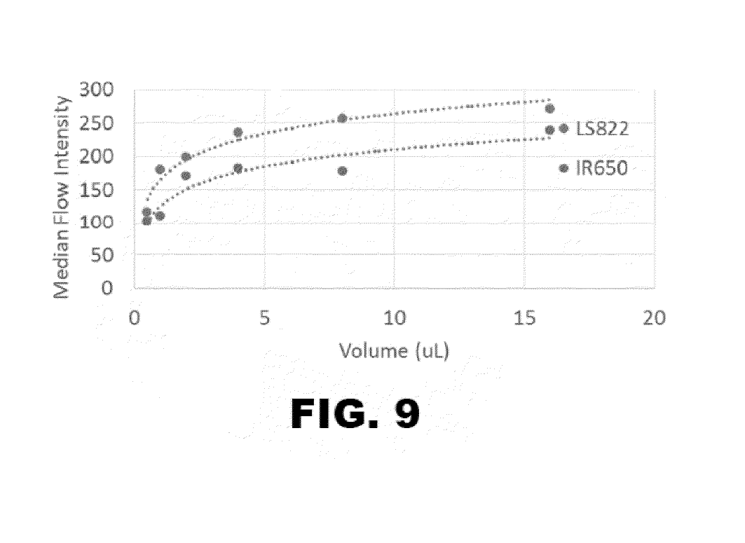

The brightness of the LS822-IgG conjugate was compared to the brightness of another conjugate IR650-IgG prepared from the commercial dye IR650 made by Li-COR (FIG. 9). LS822-IgG was 25-40% brighter. We expect that the NIR-antibody conjugates such as based on LS755 will show even stronger difference.

What is claimed is:

1. An asymmetrically charged fluorescent cyanine dye, wherein the dye comprises Formula (I): 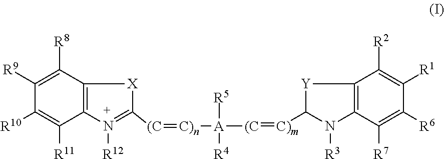
wherein:
A is selected from the group consisting of a carbon, a carbocyclic ring, and a heterocyclic ring;
X and Y are independently selected from the group consisting of a heteroatom, an alkyl group, an alkenyl group, and an alkynyl group;
n and m are independent integers from 1 to about 5;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R², R⁴, R⁵, R⁶, R⁷, R⁸, R¹⁰ and R¹¹ are independently selected from the group consisting of functional group, hydrogen, hydrocarbyl, and substituted hydrocarbyl; and
R³ and R¹² are independently selected from the group consisting of hydrocarbyl, and substituted hydrocarbyl.

2. The asymmetrically charged fluorescent cyanine dye of claim 1, wherein the dye comprises Formula (II): 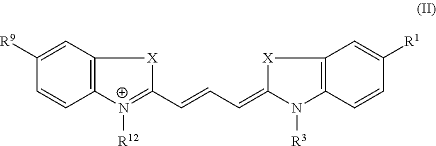
wherein:
X and Y are independently selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group; and
R³ and R¹² are independently selected from the group consisting of hydrocarbyl, and substituted hydrocarbyl.

3. The asymmetrically charged fluorescent cyanine dye of claim 1, wherein the dye comprises Formula (III): 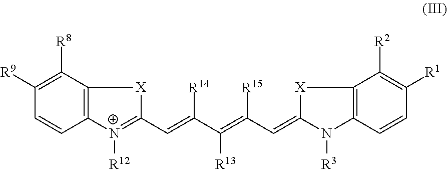
wherein:
X and Y are independently selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R² and R⁸ are independently selected from the group consisting of functional group, hydrogen, hydrocarbyl, and substituted hydrocarbyl; and
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

4. The asymmetrically charged fluorescent cyanine dye of claim 1, wherein the dye comprises Formula (IV): 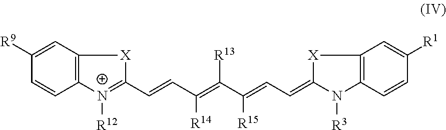
wherein:
X and Y are independently selected from the group consisting of a heteroatom, an alkyl group, an alkenyl group, and an alkynyl group;
R¹ and R⁹ are independently selected from the group consisting of a highly positive group, a highly negative group, and a neutral group with the proviso that at least one of R¹ and R⁹ is a highly positive group or a highly negative group and that R¹ and R⁹ are not both a highly positive group or both a highly negative group;
R³ and R¹² are independently selected from the group consisting of hydrocarbyl, and substituted hydrocarbyl;
R¹³ is selected from the group consisting of H, hydrocarbyl and substituted hydrocarbyl; and
R¹⁴ and R¹⁵ are either hydrogen or together form an optionally substituted carbocyclic ring or heterocyclic ring.

5. The asymmetrically charged fluorescent cyanine dye of claim 3, wherein:
R², R⁸, R¹³, R¹⁴ and R¹⁵ are H;
R³ and R¹² are (CH₂)ₙSO₃H;
n is an integer from 1 to about 5;
R¹ is a neutral group; and
R⁹ is a highly negative group.

6. The asymmetrically charged fluorescent cyanine dye of claim 4, wherein:
R¹³, R¹⁴ and R¹⁵ are H;
R³ and R¹² are (CH₂)ₙSO₃H;
n is an integer from 1 to about 5;
R¹ is a neutral group; and
R⁹ is a highly negative group.

7. A compound, the compound comprising the asymmetrically charged fluorescent cyanine dye of claim 1 conjugated to a biomolecule.

8. The compound of claim 7, wherein the biomolecule is selected from the group consisting of an antigen, an antibody, a drug, a vitamin, and a small molecule.

9. The compound of claim 7, wherein the biomolecule is selected from the group consisting of a peptide, a nucleic acid, a carbohydrate, and a lipid.

10. The compound of claim 7, wherein the biomolecule is a nanoparticle.

11. A method of use of a compound of claim 1, the method comprising: (a) administering the compound to a subject and (b) detecting the compound in the subject.

12. The method of claim 11, wherein the compound is administered intratracheally.

13. The method of claim 12, wherein the compound is detected in lung tissue.

14. The method of claim 11, wherein the compound is conjugated to a nanoparticle.

15. The method of claim 11, wherein the compound is detected about 5 min to about 24 hours after administration.

16. The method of claim 11, wherein the compound is detected about 1 day to about 7 days after administration.
